(19) United States
(12) Patent Application Publication
(10) Pub. No.: US 2025/0388662 A1
(43) Pub. Date: Dec. 25, 2025

(54) ANTIBODIES THAT BIND TO IGE-DEPENDENT HISTAMINE-RELEASING FACTOR AND USES THEREOF

(71) Applicant: EWHA UNIVERSITY - INDUSTRY COLLABORATION FOUNDATION, Seoul (KR)

(72) Inventors: Kyung Lim LEE, Seoul (KR); Hae-duck BAE, Seoul (KR); Minyoung CHO, Seoul (KR)

(73) Assignee: EWHA UNIVERSITY - INDUSTRY COLLABORATION FOUNDATION, Seoul (KR)

(21) Appl. No.: 18/881,465

(22) PCT Filed: Jul. 5, 2023
(86) PCT No.: PCT/KR2023/009523

§ 371 Date: Jan. 6, 2025

(30) Foreign Application Priority Data
Jul. 5, 2022 (KR) 10-2022-0082475

(51) Int. Cl. C07K 16/18; A61K 39/00; A61P 11/06; G01N 33/68

(52) U.S. Cl. CPC C07K 16/18; A61P 11/06; G01N 33/6893; A61K 2039/505; C07K 2317/52; C07K 2317/622; C07K 2317/92; G01N 2800/122

(57) ABSTRACT

The present invention relates to antibodies that bind to an IgE-dependent histamine-releasing factor (HRF) and uses thereof. It has been confirmed that the anti-HRF antibodies according to the present invention inhibit HRF activity by binding, with a high binding affinity, specifically to the HRF, and thus can be usefully employed to develop agents for preventing and treating HRF-related diseases such as allergic diseases, inflammatory diseases, autoimmune diseases, cancer, hypertension, malaria, or osteoporosis.

### TECHNICAL FIELD

The present invention relates to antibodies that bind to IgE-dependent histamine releasing factor (HRF) and uses thereof.

### BACKGROUND ART

Histamine-releasing factor (HRF, Translationally Controlled Tumor Protein, TCTP, hereinafter referred to as HRF) is known to activate basophils and induce the release of histamine, which triggers a late allergic response, and also activates inflammatory cells involved in late allergic response in addition to basophils and releases various cytokines, and thus it is recognized as an important factor in the late allergic response (MacDonald et al., Science, 269, 688-690, 1995).

HRF was known to induce the release of histamine from basophils in the presence of specific IgE, but was subsequently observed to regulate the secretion of histamine, IL-4, and IL-13 from inflammatory cells in the presence or absence of IgE and its receptor, FcER, leading to the possibility that HRF acts by binding to a specific cell membrane receptor rather than IgE by Bheekha-Escura et al.

Through prior research, the present inventors identified that HRE, which can form dimers, secretes histamine and IL-8 in cells, demonstrated for the first time that the dimerized form of HRF is an allergenic substance (Korean Patent No. 100780255, European Patent No. 1683866, Japanese Patent No. 4564926, and U.S. Pat. No. 7,772,368), and also first discovered that HRF can cross the cell membrane despite HRF being a hydrophilic protein and that HRF inside the cell binds to the large cytoplasmic loop CD3 (cytoplasmic domain 3) of the Na, K-ATPase alpha subunit, and demonstrated that the overexpression of intracellular HRF in transgenic mice induces hypertension (Korean Patent No. 10-0457350, European Patent No. 1167526, Japanese Patent No. 4295449, and U.S. Pat. No. 6,710,165). In addition, a peptide having a membrane-penetrating protein domain function was discovered in HRF (Korean Patent No. 10-0859972). In addition, the present inventors independently developed a peptide drug (dTBP2) with anti-allergic efficacy by targeting and controlling dimeric HRF, and demonstrated inhibitory efficacy in allergic diseases and rheumatoid arthritis (Korean Patent No. 10-1830838). In addition, as HRF structural parts, the flexible loop domain (Korean Patent No. 101804291) or helix 2 domain (Korean Patent No. 101843051) and C-terminus (Korean Patent No. 101804285) that bind to HRF receptors present in the cell membrane were identified, and it was confirmed that binding substances to theminhibit the secretion ability of IL-8. In addition, it was found that, as anti-allergic agents, dehydrocostus lactone (Phytomedicine 2018), and cardamonin (FRONTIERS IN PHARMACOLOGY, 2021, v. 12, 765521) exerted anti-inflammatory effects by inhibiting dimeric HRF, and confirmed that monomeric and dimeric HRF are closely related to disease activity in rheumatoid arthritis and that HRF is a novel biomarker and therapeutic target for the diagnosis and treatment of rheumatoid arthritis (EXPERIMENTAL AND MOLECULAR MEDICINE, 2021, 67-80). Furthermore, it was identified that dTBP2, an inhibitor of dimeric HRF, directly inhibits degranulation of mast cells, attenuating the systemic anaphylactic reaction (FRONTIERS IN PHARMACOLOGY, 2021, v. 12, 764321), HRF may be a therapeutic target for obesity and obesity-related metabolic disorders, including type 2 diabetes (International Journal of Obesity, 2021, v. 45 no. 7, 1576-1587), and dimeric HRF exacerbates airway inflammation through activation of airway epithelial cells (Biomedicine and Pharmacotherapy, 2021, v. 144, 112316).

Thus, the present inventors have endeavored to develop antibodies that specifically bind to HRFs that are associated with various diseases as described above, and in particular have developed antibodies that bind to monomeric and/or dimeric HRFs. The present inventors confirmed that the anti-HRF antibody according to the present invention inhibits the cytokine-like activity of HRF by specifically binding to HRF with high binding affinity, discovered that the anti-HRF antibody can be usefully employed in the development of agents for preventing and treating HRF-related diseases such as allergic diseases, chronic inflammatory diseases, autoimmune diseases, or cancer, hypertension, malaria, osteoporosis, and completed the present invention.

### DISCLOSURE

### Technical Problem

An object of the present invention is to provide an antibody or antigen-binding fragment thereof that specifically binds to monomeric and/or dimeric histamine-releasing factor (HRF).

Another object of the present invention is to provide a polynucleotide encoding the antibody or antigen-binding fragment thereof.

Another object of the present invention is to provide an expression vector comprising the polynucleotide.

Another object of the present invention is to provide a transformant into which the expression vector is introduced.

Another object of the present invention is to provide a pharmaceutical composition for preventing or treating HRF-related diseases, comprising the antibody or antigen-binding fragment thereof.

Another object of the present invention is to provide a composition for diagnosing HRF-related diseases, comprising the antibody or antigen-binding fragment thereof.

Another object of the present invention is to provide a kit for diagnosing HRF-related diseases, comprising the antibody or antigen-binding fragment thereof.

Another object of the present invention is to provide a kit for detecting HRF, comprising the composition comprising the antibody or antigen-binding fragment thereof.

Another object of the present invention is to provide a method for diagnosing HRF-related diseases, using the antibody or antigen-binding fragment thereof.

### Technical Solution

In order to achieve the above objects, the present invention provides an antibody or antigen-binding fragment thereof that specifically binds to monomeric and/or dimeric histamine-releasing factor (HRF), comprising: CDR sequences of a variable light chain domain (VL) or a variable heavy chain domain (VH) selected from the group consisting of:
(i) a light chain CDR1 set forth in SEQ ID NO: 2; a light chain CDR2 set forth in SEQ ID NO: 3; a light chain CDR3 set forth in SEQ ID NO: 4; a heavy chain CDR1 set forth in SEQ ID NO: 6; a heavy chain CDR2 set forth in SEQ ID NO: 7; a heavy chain CDR3 set forth in SEQ ID NO: 8;
(ii) a light chain CDR1 set forth in SEQ ID NO: 12; a light chain CDR2 set forth in SEQ ID NO: 13; a light chain CDR3 set forth in SEQ ID NO: 14; a heavy chain CDR1 set forth in SEQ ID NO: 16; a heavy chain CDR2 set forth in SEQ ID NO: 17; a heavy chain CDR3 set forth in SEQ ID NO: 18;
(iii) a light chain CDR1 as set forth in SEQ ID NO: 22; a light chain CDR2 as set forth in SEQ ID NO: 23; a light chain CDR3 as set forth in SEQ ID NO: 24; a heavy chain CDR1 as set forth in SEQ ID NO: 26; a heavy chain CDR2 as set forth in SEQ ID NO: 27; a heavy chain CDR3 as set forth in SEQ ID NO: 28;
(iv) a light chain CDR1 set forth in SEQ ID NO: 32; a light chain CDR2 set forth in SEQ ID NO: 33; a light chain CDR3 set forth in SEQ ID NO: 34; a heavy chain CDR1 set forth in SEQ ID NO: 36; a heavy chain CDR2 set forth in SEQ ID NO: 37; a heavy chain CDR3 set forth in SEQ ID NO: 38;
(v) a light chain CDR1 set forth in SEQ ID NO: 42; a light chain CDR2 set forth in SEQ ID NO: 43; a light chain CDR3 set forth in SEQ ID NO: 44; a heavy chain CDR1 set forth in SEQ ID NO: 46; a heavy chain CDR2 set forth in SEQ ID NO: 47; a heavy chain CDR3 set forth in SEQ ID NO: 48;
(vi) a light chain CDR1 set forth in SEQ ID NO: 52; a light chain CDR2 set forth in SEQ ID NO: 53; a light chain CDR3 set forth in SEQ ID NO: 54; a heavy chain CDR1 set forth in SEQ ID NO: 56; a heavy chain CDR2 set forth in SEQ ID NO: 57; a heavy chain CDR3 set forth in SEQ ID NO: 58;
(vii) a light chain CDR1 set forth in SEQ ID NO: 62; a light chain CDR2 set forth in SEQ ID NO: 63; a light chain CDR3 set forth in SEQ ID NO: 64; a heavy chain CDR1 set forth in SEQ ID NO: 66; a heavy chain CDR2 set forth in SEQ ID NO: 67; a heavy chain CDR3 set forth in SEQ ID NO: 68; and
(viii) a light chain CDR1 set forth in SEQ ID NO: 72; a light chain CDR2 set forth in SEQ ID NO: 73; a light chain CDR3 set forth in SEQ ID NO: 74; a heavy chain CDR1 set forth in SEQ ID NO: 76; a heavy chain CDR2 set forth in SEQ ID NO: 77; a heavy chain CDR3 set forth in SEQ ID NO: 78.

In addition, the present invention provides a polynucleotide encoding the antibody or antigen-binding fragment thereof.

In addition, the present invention provides an expression vector comprising the polynucleotide.

In addition, the present invention provides a transformant into which the expression vector is introduced.

In addition, the present invention provides a pharmaceutical composition for preventing or treating HRF-related diseases, comprising the antibody or antigen-binding fragment thereof.

In addition, the present invention provides a composition for diagnosing HRF-related diseases, comprising the antibody or antigen-binding fragment thereof.

In addition, the present invention provides a kit for diagnosing HRF-related diseases, comprising the antibody or antigen-binding fragment thereof.

In addition, the present invention provides a kit for detecting HRF, comprising a composition comprising the antibody or antigen-binding fragment thereof.

In addition, the present invention provides a method for diagnosing HRF-related diseases, using the antibody or antigen-binding fragment thereof.

### Advantageous Effects

The present invention relates to antibodies that bind to an IgE-dependent histamine-releasing factor (HRF) and uses thereof. It has been confirmed that the anti-HRF antibodies according to the present invention inhibit the cytokine-like activity of HRF by binding, with a high binding affinity, specifically to the HRF, and thus can be usefully employed to develop agents for preventing and treating HRF-related diseases such as allergic diseases, chronic inflammatory diseases, autoimmune diseases, or cancer, hypertension, malaria, osteoporosis.

### DESCRIPTION OF DRAWINGS

FIG. 1a is a schematic diagram of an expression vector constructed to produce a recombinant HRF protein, i.e., an antigen protein, used in the production of anti-HRF monoclonal antibodies of the present invention.

FIG. 1b shows the structure of HRF monomer.

FIG. 1c shows the structure of HRF dimer.

FIG. 1d shows the expression and purification of the HRF monomer confirmed by SDS-PAGE.

FIG. 1e shows the expression and purification of the HRF dimer confirmed by SDS-PAGE.

FIG. 2a shows a panning assay utilized in the antibody screening process of the present invention.

FIG. 2b shows 38 clones that tested positive for HRF dimer out of 288 clones in a phage ELISA performed after panning from the scFv phage library.

FIG. 2c shows 33 clones that tested positive for HRF monomer out of 288 clones in a phage ELISA performed after panning.

FIG. 2d shows the amino acid sequences of anti-HRF/TCTP dimer scFv antibodies.

FIG. 2e shows the amino acid sequences of anti-HRF/TCTP monomer scFv antibodies.

FIG. 3a shows the results of SDS-PAGE performed under non-reducing conditions after production of anti-HRF ScFv-Fc antibodies.

FIG. 3b shows the results of SDS-PAGE performed under reducing conditions after production of anti-HRF scFv-Fc antibodies.

FIG. 4a shows a comparative analysis of the binding affinity of eight anti-HRF scFv-Fc antibodies and antibodies that bind to human HRF dimer.

FIG. 4b shows a comparative analysis of the binding affinity of anti-HRF scFv-Fc antibodies and antibodies that bind to mouse/rat HRF dimer.

FIG. 5 shows the IL-8 secretion inhibition ability of anti-HRF scFv-Fc antibodies in bronchial epithelial cells.

FIG. 6 shows the method for producing an asthma mouse model and the timing of anti-HRF SCFV-Fc antibody administration.

FIG. 7 shows the results of measuring the total leukocyte count in the bronchoalveolar lavage fluid of asthmatic mice.

FIG. 8 shows the results of measuring the production of IL-4, IL-5, and IL-13 in the bronchoalveolar lavage fluid of asthmatic mice.

FIG. 9 shows the results of measuring the amount of ovalbumin-specific IgE produced in the plasma of asthmatic mice.

FIG. 10 shows the results of analyzing the lung tissue of asthmatic mice.

### BEST MODE

Hereinafter, the present invention will be described in more detail.

The present invention provides an antibody or antigen-binding fragment thereof that specifically binds to monomeric and/or dimeric histamine-releasing factor (HRF).

The HRF is an IgE-dependent histamine releasing factor with histamine releasing activity, and is a well-known protein consisting of 172 amino acids present in all cytoplasm, known as translationally controlled tumor protein (TCTP). The HRF has been reported to cause late-response allergic diseases such as allergic rhinitis, asthma, and atopic dermatitis by stimulating IgE-sensitized basophils to promote the release of histamine and interleukin-4 (IL-4).

The HRF is characterized in that the dimer is the active form and the flexible loop (FL) domain or the helix 2 (H2) domain of the HRF structure is a site that binds to the receptor for HRF, and the antibody that specifically binds to the HRF is preferably one that binds to any one selected from the group consisting of those having a full-length and an N-terminal truncated form. In an embodiment of the present invention, the dimeric HRF has 10 amino acids truncated from the N-terminus. In an embodiment of the present invention, the monomeric or dimeric HRF may comprise an amino acid sequence of SEQ ID NO: 82 or SEQ ID NO: 83, respectively.

Specifically, the antibody or antigen-binding fragment thereof that specifically binds to the monomeric and/or dimeric HRF of the present invention may be selected from the group consisting of the following (1) to (8):
(1) an antibody or antigen-binding fragment thereof comprising a light chain CDR1 set forth in SEQ ID NO: 2; a light chain CDR2 set forth in SEQ ID NO: 3; a light chain CDR3 set forth in SEQ ID NO: 4; a heavy chain CDR1 set forth in SEQ ID NO: 6; a heavy chain CDR2 set forth in SEQ ID NO: 7; a heavy chain CDR3 set forth in SEQ ID NO: 8;
(2) an antibody or antigen-binding fragment thereof comprising a light chain CDR1 set forth in SEQ ID NO: 12; a light chain CDR2 set forth in SEQ ID NO: 13; a light chain CDR3 set forth in SEQ ID NO: 14; a heavy chain CDR1 set forth in SEQ ID NO: 16; a heavy chain CDR2 set forth in SEQ ID NO: 17; a heavy chain CDR3 set forth in SEQ ID NO: 18;
(3) an antibody or antigen-binding fragment thereof comprising a light chain CDR1 as set forth in SEQ ID NO: 22; a light chain CDR2 as set forth in SEQ ID NO: 23; a light chain CDR3 as set forth in SEQ ID NO: 24; a heavy chain CDR1 as set forth in SEQ ID NO: 26; a heavy chain CDR2 as set forth in SEQ ID NO: 27; a heavy chain CDR3 as set forth in SEQ ID NO: 28;
(4) an antibody or antigen-binding fragment thereof comprising a light chain CDR1 set forth in SEQ ID NO: 32; a light chain CDR2 set forth in SEQ ID NO: 33; a light chain CDR3 set forth in SEQ ID NO: 34; a heavy chain CDR1 set forth in SEQ ID NO: 36; a heavy chain CDR2 set forth in SEQ ID NO: 37; a heavy chain CDR3 set forth in SEQ ID NO: 38;
(5) an antibody or antigen-binding fragment thereof comprising a light chain CDR1 set forth in SEQ ID NO: 42; a light chain CDR2 set forth in SEQ ID NO: 43; a light chain CDR3 set forth in SEQ ID NO: 44; a heavy chain CDR1 set forth in SEQ ID NO: 46; a heavy chain CDR2 set forth in SEQ ID NO: 47; a heavy chain CDR3 set forth in SEQ ID NO: 48;
(6) an antibody or antigen-binding fragment thereof comprising a light chain CDR1 set forth in SEQ ID NO: 52; a light chain CDR2 set forth in SEQ ID NO: 53; a light chain CDR3 set forth in SEQ ID NO: 54; a heavy chain CDR1 set forth in SEQ ID NO: 56; a heavy chain CDR2 set forth in SEQ ID NO: 57; a heavy chain CDR3 set forth in SEQ ID NO: 58;
(7) an antibody or antigen-binding fragment thereof comprising a light chain CDR1 set forth in SEQ ID NO: 62; a light chain CDR2 set forth in SEQ ID NO: 63; a light chain CDR3 set forth in SEQ ID NO: 64; a heavy chain CDR1 set forth in SEQ ID NO: 66; a heavy chain CDR2 set forth in SEQ ID NO: 67; a heavy chain CDR3 set forth in SEQ ID NO: 68; and
(8) an antibody or antigen-binding fragment thereof comprising a light chain CDR1 set forth in SEQ ID NO: 72; a light chain CDR2 set forth in SEQ ID NO: 73; a light chain CDR3 set forth in SEQ ID NO: 74; a heavy chain CDR1 set forth in SEQ ID NO: 76; a heavy chain CDR2 set forth in SEQ ID NO: 77; a heavy chain CDR3 set forth in SEQ ID NO: 78.

In another embodiment, the antibody or antigen-binding fragment thereof that specifically binds to the monomeric and/or dimeric HRF of the present invention may be selected from the group consisting of the following (9) to (16):
(9) an antibody or antigen-binding fragment thereof comprising a light chain variable region set forth in SEQ ID NO: 1 and a heavy chain variable region set forth in SEQ ID NO: 5;
(10) an antibody or antigen-binding fragment thereof comprising a light chain variable region set forth in SEQ ID NO: 11 and a heavy chain variable region set forth in SEQ ID NO: 15;
(11) an antibody or antigen-binding fragment thereof comprising a light chain variable region set forth in SEQ ID NO: 21 and a heavy chain variable region set forth in SEQ ID NO: 25;
(12) an antibody or antigen-binding fragment thereof comprising a light chain variable region set forth in SEQ ID NO: 31 and a heavy chain variable region set forth in SEQ ID NO: 35;
(13) an antibody or antigen-binding fragment thereof comprising a light chain variable region set forth in SEQ ID NO: 41 and a heavy chain variable region set forth in SEQ ID NO: 45;
(14) an antibody or antigen-binding fragment thereof comprising a light chain variable region set forth in SEQ ID NO: 51 and a heavy chain variable region set forth in SEQ ID NO: 55;
(15) an antibody or antigen-binding fragment thereof comprising a light chain variable region set forth in SEQ ID NO: 61 and a heavy chain variable region set forth in SEQ ID NO: 65;
(16) an antibody or antigen-binding fragment thereof comprising a light chain variable region set forth in SEQ ID NO: 71 and a heavy chain variable region set forth in SEQ ID NO: 75.

In another embodiment, the antibody or antigen-binding fragment thereof that specifically binds to the monomeric and/or dimeric HRF of the present invention may be selected from the group consisting of the following (17) to (24):
(17) an antibody or antigen-binding fragment thereof comprising the amino acid sequence set forth in SEQ ID NO: 9;
(18) an antibody or antigen-binding fragment thereof comprising the amino acid sequence set forth in SEQ ID NO: 19;
(19) an antibody or antigen-binding fragment thereof comprising the amino acid sequence set forth in SEQ ID NO: 29;
(20) an antibody or antigen-binding fragment thereof comprising the amino acid sequence set forth in SEQ ID NO: 39;
(21) an antibody or antigen-binding fragment thereof comprising the amino acid sequence set forth in SEQ ID NO: 49;
(22) an antibody or antigen-binding fragment thereof comprising the amino acid sequence set forth in SEQ ID NO: 59;
(23) an antibody or antigen-binding fragment thereof comprising the amino acid sequence set forth in SEQ ID NO: 69; and
(24) an antibody or antigen-binding fragment thereof comprising the amino acid sequence set forth in SEQ ID NO: 79.

In the present invention, an antibody refers to a polypeptide that specifically binds to and recognizes an antigen, such as a monomeric and/or dimeric HRF protein. The present invention comprises not only a complete antibody form that binds to monomeric and/or dimeric HRF protein, but also antigen-binding fragments of the antibody molecule.

In the present invention, the antibody may include all of monoclonal antibodies, polyclonal antibodies, and multispecific antibodies (e.g., bispecific antibodies). The antibody comprises two heavy chains and two light chains, and has a variable region whose amino acid sequence varies depending on the type of target antigen, and a constant region whose sequence remains unchanged.

The antibody or antigen-binding fragment thereof may be a full-length antibody or an antibody fragment. Preferably, the antibody in the present invention may be a monoclonal antibody.

The antibody of the present invention may be a human antibody, a humanized antibody, or a chimeric antibody.

The term “human antibody” or “humanized antibody” as used herein refers to an antibody produced by a human or a human cell, or an antibody that possesses an amino acid sequence corresponding to the amino acid sequence of an antibody derived from a non-human source that utilizes the human antibody repertoire or another human antibody coding sequence.

The term “chimeric antibody” as used herein refers to an antibody in which a portion of the heavy chain and/or light chain is derived from a particular source or species, and the remainder of the heavy chain and/or light chain is derived from a different source or species.

The antigen-binding fragment may be single-chain variable fragment (scFv), Fab, Fab′, F(ab′)₂, Fd, and Fv. In an embodiment, the antigen-binding fragment is an antigen-binding fragment in the form of SCFV-FC (Fragment, crystallizable).

The scFv refers to a protein in which the variable domains of the light and heavy chains of an antibody are connected by a linker consisting of a peptide chain having about 15 amino acids. Light chain variable domain (VL)-linker-light chain variable domain (VH), or heavy chain variable domain (VH)-linker-light chain variable domain (VL) sequences are all possible, with the same or similar antigenic specificity as the original antibody.

In the antigen-binding fragment of an antibody to monomeric and/or dimeric HRF, e.g., an anti-HRF SCFv, the heavy chain variable region and light chain variable region may be linked with or without a linker, e.g., a peptide linker. The peptide linker may be a polypeptide consisting of from 1 to 100 or from 2 to 50 any amino acids, and the types of amino acids contained therein are not limited. The peptide linker may comprise, for example, Gly, Asn, and/or Ser residues, and may also comprise neutral amino acids such as Thr and/or Ala. Amino acid sequences suitable for the peptide linker are known in the art. Meanwhile, the linker may vary in length, as long as it does not affec the function of the bispecific antibody. For example, the peptide linker may comprise at least one selected from the group consisting of Gly, Asn, Ser, Thr, and Ala, in a total of 1 to 100, from 2 to 50, or from 5 to 25. In an embodiment, the peptide linker is an 18-mer linker, GGSSRSSSSGGGGSGGGG (SEQ ID NO: 81).

The antibody or antigen-binding fragment thereof of the present invention may comprise a variant of the amino acid sequence listed in the attached sequence list within the scope capable of specifically recognizing the HRF protein. For example, the amino acid sequence of an antibody may be changed to improve the binding affinity and/or other biological properties of the antibody. Such modifications include, for example, deletions, insertions, and/or substitutions of amino acid sequence residues of the antibody.

These amino acid modifications are made based on the relative similarity of the amino acid side chain substituents, e.g., hydrophobicity, hydrophilicity, charge, size, etc. According to analysis of the size, shape and type of amino acid side chain substitutions, it may be seen that arginine, lysine and histidine are all positively charged residues; alanine, glycine and serine have similar sizes; and phenylalanine, tryptophan, and tyrosine have similar shapes. Therefore, based on these considerations, arginine, lysine and histidine; alanine, glycine and serine; and phenylalanine, tryptophan, and tyrosine may be biologically functional equivalents.

Amino acid exchanges in proteins that do not alter the overall activity of the molecule are known in the art (H. Neurath, R. L. Hill, The Proteins, Academic Press, New York, 1979). The most common exchanges are between amino acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, or Asp/Gly.

Further, the present invention also provides a polynucleotide encoding the antibody or antigen-binding fragment thereof.

It is contemplated that the polynucleotide encoding the antibody or antigen-binding fragment thereof of the present invention also includes a polynucleotide base sequence showing substantial identity to the polynucleotide base sequence described above. The substantial identity means the sequence exhibiting at least 80% homology, more preferably at least 90% homology, most preferably at least 95% homology, when the sequence of the present invention and any other sequence are aligned to correspond as much as possible and the aligned sequences are analyzed using algorithms commonly used in the art. Alignment methods for sequence comparison are known in the art. Various methods and algorithms for alignment are disclosed in the documents [Smith and Waterman, Adv. Appl. Math. 2:482 (1981)]; [Needleman and Wunsch, J. Mol. Bio. 48:443 (1970)]; [Pearson and Lipman, Methods inMol. Biol. 24:307-31 (1988)]; [Higgins and Sharp, Gene 73:237-44 (1988)]; [Higgins and Sharp, CABIOS 5:151-3 (1989)]; [Corpet et al., Nuc. Acids Res. 16:10881-90 (1988)]; [Huang et al., Comp. Appl. BioSci. 8:155-65 (1992)], and [Pearson et al., Meth. Mol. Biol. 24:307-31 (1994)], etc. The NCBI Basic Local Alignment Search Tool (BLAST) (Altschul et al., J. Mol. Biol. 215:403-10 (1990)) is accessible from the National Center for Biological Information (NBCI) and elsewhere, and is available in conjunction with sequence analysis programs such as blastp, blastn, blastx, tblastn, and tblastx on the Internet. BLAST is accessible through the BLAST page on the ncbi website. A method for comparing sequence homology using this program can be found on the BLAST help page on the ncbi website.

In an embodiment, the antibody or antigen-binding fragment thereof that specifically binds to the monomeric and/or dimeric HRF of the present invention may be selected from the group consisting of the following (25) to (32):
(25) an antibody or antigen-binding fragment thereof comprising the amino acid sequence encoded by the nucleotide sequence set forth in SEQ ID NO: 10;
(26) an antibody or antigen-binding fragment thereof comprising the amino acid sequence encoded by the nucleotide sequence set forth in SEQ ID NO: 20;
(27) an antibody or antigen-binding fragment thereof comprising the amino acid sequence encoded by the nucleotide sequence set forth in SEQ ID NO: 30;
(28) an antibody or antigen-binding fragment thereof comprising the amino acid sequence encoded by the nucleotide sequence set forth in SEQ ID NO: 40;
(29) an antibody or antigen-binding fragment thereof comprising the amino acid sequence encoded by the nucleotide sequence set forth in SEQ ID NO: 50;
(30) an antibody or antigen-binding fragment thereof comprising the amino acid sequence encoded by the nucleotide sequence set forth in SEQ ID NO: 60;
(31) an antibody or antigen-binding fragment thereof comprising the amino acid sequence encoded by the nucleotide sequence set forth in SEQ ID NO: 70;
(32) an antibody or antigen-binding fragment thereof comprising the amino acid sequence encoded by the nucleotide sequence set forth in SEQ ID NO: 80.

In addition, the present invention provides an expression vector comprising the polynucleotide.

The vector may be capable of replicating and/or expressing the polynucleotide in a cell. The cell may be a eukaryotic cell or a prokaryotic cell. The eukaryotic cell may be a mammalian cell, a plant cell, a yeast cell, or an insect cell. The mammal may be a human, a monkey, a rabbit, a rat, a hamster, or a mouse. The prokaryotic cell may be a bacterial cell. The bacteria may be Escherichia coli. The vector may be an expression vector. The expression vector may be one in which the polynucleotide is operably linked to an appropriate regulatory region such that the polynucleotide can be expressed in a host cell. The regulatory region may be a promoter, an enhancer, or a terminator. The vector may also include a selection marker. The vector may be a phage, plasmid, cosmid, mini-chromosome, virus, or retroviral vector. The vector may comprise a polynucleotide encoding a heavy chain variable region or a light chain variable region of the antibody, respectively, or may comprise both polynucleotides encoding a heavy chain variable region or a light chain variable region thereof.

The recombinant vector system of the present invention can be constructed by various methods known in the art.

Antibodies specific for the HRF surface antigen may preferably be selected by applying phage-display technology (Smith, Science, 228, 1315-1317, 1985; and Hoogenboom & Chames, Immunol Today, 21, 371-378, 2000). By applying phage-display technology as follows, a gene expressing a desired antibody may be fused to a gene (gene III) expressing a filamentous phage (M13, Fd and F1) coat protein, thereby generating a virus particle in the form of an antibody-phage in which the fused antibody is exposed on the surface of a bacteriophage particle, and high specificity and affinity of the exposed antibody and the high infectivity of the phage may be utilized to select a desired antibody from the phage library by applying the biopanning assay (Burton & Barbas, Adv. Immunol., 57, 191-280, 1994; Winter et al., Annu. Rev. Immunol., 12, 433-455, 1994; and Hoogenboom et al., Immunotechnology, 4, 1-20, 1998; Kim et al., Hybrid Hybridomics, 21, 385-392, 2002).

In addition, the present invention provides a transformant into which the expression vector is introduced.

The transformant into which the expression vector is introduced provided in the present invention is not particularly limited, but may be a bacterial cell such as Escherichia coli, Streptomyces, or Salmonella typhimurium; a yeast cell; a fungal cell such as Pichia pastoris; an insect cell such as Drosophila or Spodoptera Sf9 cell; an animal cell such as CHO (Chinese hamster ovary cell), SP2/0 (mouse myeloma), human lymphoblastoid, COS, NSO (mouse myeloma), 293T, Bow melanoma cell, HT-1080, BHK (baby hamster kidney cell), HEK (human embryonic kidney cell), PERC. 6 (human retinal cell), or a plant cell, transformed by the introduction of the expression vector.

As used herein, the term “introduction” refers to a method of delivering a vector comprising a polynucleotide encoding the antibody or antigen-binding fragment thereof to a host cell. The introduction may be performed by any of several methods known in the art, such as calcium phosphate-DNA co-precipitation, DEAE-dextran-mediated transfection, transfection, electroporation, polybrene-mediated microinjection, liposome fusion, lipofectamine, and protoplast fusion.

Further, the present invention provides a composition for preventing or treating HRF-related diseases, comprising the antibody or antigen-binding fragment thereof.

The HRF-related disease may be selected from the group consisting of inflammatory diseases, autoimmune diseases, cancer, hypertension, malaria, and osteoporosis.

The inflammatory disease or autoimmune disease may be selected from the group consisting of asthma, bronchitis, chronic obstructive pulmonary diseases, bronchiectasis, rhinitis, atopic dermatitis, urticaria, hay fever, conjunctivitis, allergic diseases such as anaphylaxis, bronchitis, pneumonia, arthritis, nephritis, psoriasis, dermatitis, Crohn's disease, enteritis, gingivitis, arteriosclerosis, coronary arteritis, hepatitis, Behcet's disease, bladder cancer, prostatitis, pyelonephritis, glomerulonephritis, osteomyelitis, thyroiditis, uveitis, peritonitis, meningitis, pulmonary fibrosis, and rheumatoid arthritis.

The cancer may be selected from the group consisting of oral cancer, liver cancer, stomach cancer, colon cancer, breast cancer, lung cancer, bone cancer, pancreatic cancer, skin cancer, head and neck cancer, skin cancer, cervical cancer, ovarian cancer, colorectal cancer, small intestine cancer, rectal cancer, fallopian tube carcinoma, perianal cancer, endometrial carcinoma, vaginal carcinoma, vulvar carcinoma, Hodgkin's disease, esophageal cancer, lymphoma, bladder cancer, gallbladder cancer, endocrine cancer, thyroid cancer, parathyroid cancer, adrenal cancer, soft tissue sarcoma, urethral cancer, penile cancer, prostate cancer, chronic leukemia, acute leukemia, lymphocytic lymphoma, kidney cancer, ureteral cancer, renal cell carcinoma, pelvic carcinoma, central nervous system tumor, primary central nervous system lymphoma, spinal cord tumor, brainstem glioma, and pituitary adenoma.

IL-8 is known to be involved in various inflammatory diseases such as chronic inflammatory bronchial diseases such as chronic bronchitis (Richman-Eisenstat et al., Am J Physiol, 264, L413-418, 1993), inflammatory lung diseases such as pneumonia (Erger and Casale, Eur Respir J, 11, 299-305, 1998; Pease & Sabroe, Am J Respir Med, 1, 19-25, 2002), arthritis or nephritis (Harada et al., J Leukoc Biol, 56, 559-564, 1994), psoriasis (Schulz et al., J Immunol, 151, 4399-4406, 1993; Bruch-Gerharz et al., J Exp Med, 184, 2007-2012, 1996), dermatitis (Sticherling et al., Arch Dermatol Res, 284, 82-85, 1992), Crohn's disease (Izutani et al., Inflamm Bowel Dis, 1, 37-47, 1995), inflammatory bowel disease (Mitsuyama et al., Clin Exp Immunol, 96, 432-436, 1994), gingivitis (Haake & Huang, Clinical Periodontology, 9th Edition. Philadelphia: W.B. Saunders Co. 2002. page 162), cardiovascular diseases such as arteriosclerosis and coronary artery disease (Apostolakis et al., Cardiovasc Res, 84, 353-360, 2009; Boekholdt et al., Arterioscler Thromb Vasc Biol, 24, 1503-1508, 2004), chronic liver disease (Zimmermann et al., PLOS ONE, 6, e21381, 2011), Behcet's disease (Katsantonis et al., Dermatology, 201, 37-39, 2000), bladder cancer, prostatitis, pyelonephritis or osteomyelitis (Shahzad et al., Int arch med, 3, 11, 2010), thyroid disease (Kobawala et al., J Thyroid Res, 8, 270-149, 2011), uveitis (Klok et al., Br J Ophthalmol, 82, 871-874, 1998), glomerulonephritis, peritonitis, meningitis, and pulmonary fibrosis (Harada et al., Mol Med Today, 2, 482-489, 1996). Therefore, inhibition of IL-8 has been suggested as a therapeutic strategy in inflammatory diseases such as lung disease, rheumatoid arthritis, inflammatory bowel disease, psoriasis, chronic inflammatory skin diseases such as palmoplantar pustulosis, and ocular inflammation (Mukaida, Am J Physiol Lung Cell Mol Physiol, 284, L566-L577, 2003; Skov et al., J Immunol, 181, 669-679, 2008; Harada et al., J Leukoc Biol, 56, 559-564, 1994). Blocking antibodies to IL-8 or treatments that inhibit the gene encoding the IL-8 receptor are effective in treating inflammation (Harada et al., Mol Med Today, 2, 482-489, 1996), and for example, administration of antibodies to IL-8 reduced inflammation in patients with chronic inflammatory skin diseases (Skov et al., J Immunol, 181, 669-679, 2008). GM-CSF, the secretion of which is increased by HRF, has also been implicated in a variety of inflammatory diseases (Hamilton, Trends Immunol, 23, 403-408, 2002), and GM-CSF has also been proposed as a target for inflammatory diseases such as rheumatoid arthritis (Cornish et al., Nat Rev Rheumatol, 5, 554-559, 2009). Thus, by confirming that HRF activity inhibitors are involved in the inhibition of IL-8 secretion, the theory that the HRF activity inhibitors of the present invention can prevent and treat the above diseases has been shown to be certain. Thus, the HRF antibody of the present invention may be utilized in pharmaceutical compositions for the prevention and treatment of inflammatory diseases.

In addition, IL-8, which is increased by HRF, is known to be involved in various allergic diseases such as asthma or bronchitis (Chanez et al., Int Arch Allergy Immunol, 111, 83-88, 1996), chronic obstructive pulmonary disease (Nocker et al., Int Arch Allergy Immunol, 109, 183-191, 1996), bronchiectasis (Simpson et al., Thorax, 62, 211-218, 2007), rhinitis (Benson et al., Pediatr Allergy Immunol, 10, 178-185, 1999; Kuna et al., J Allergy Clin Immun, 97, 104-112, 1996), atopic dermatitis (Kimata & Lindley, Arch Dis Child 70,119-122, 1994), urticaria (Choi et al., J Clin Immunol, 28, 244-249, 2008), hay fever (Ciprandi et al., Otolaryngol Head Neck Surg, 133, 429-435, 2005), conjunctivitis (Miyoshi et al., Cornea, 20, 743-747, 2001), and anaphylaxis. Thus, the HRF antibody of the present invention may be utilized in pharmaceutical compositions for the prevention and treatment of allergic diseases.

Further, HRF has been known as a tumor-specific protein until the 1980s, and its synthesis was thought to be related to the proliferative stage of tumors. In mouse erythroleukemia cells, it was identified as the 21 kDa oncoprotein p21 (Chitpatima et al, 1988), and in Ehrlich ascites tumor, the protein p23, which is associated with cell growth, was found to be identical to HRF (Bohm et al, 1989). Thus, the HRF antibody of the present invention may be utilized in pharmaceutical compositions for the prevention and treatment of cancer.

HRF is also known to cause hypertension by inhibiting sodium/potassium APTase pump action, thereby affecting the responsiveness and contractility of vascular smooth muscle and heart muscle, and neurotransmitter secretion in the brain (Korean Patent Laid-Open Publication No. 10-2004-0111051). Thus, the HRF antibody of the present invention may be utilized in pharmaceutical compositions for the prevention and treatment of hypertension.

In addition, in 1998, the anti-malarial drug Artemisinin was shown to work by binding to the malaria protein HRF (Bhisutthibhan et al., J Biol Chem, 273, 16192-16198, 1998). IL-8 is also secreted by malaria patients (Friedland et al., Trans R Soc Trop Med Hyg, 87, 54-55, 1993), and it has been reported that HRF in malaria promotes IL-8 secretion (MacDonald et al., Proc Natl Acad Sci USA, 98, 10829-32, 2001). Thus, the HRF antibody of the present invention may be utilized in pharmaceutical compositions for the prevention and treatment of malaria.

Further, it is known that HRF is induced during the differentiation of osteoclasts, which destroy bone, and that mice with overexpression of HRF have reduced bone mass and increased osteoclasts (SW Choi et al., FEBS letters, 588 (21), 4026-31, 2004). Thus, the HRF antibody of the present invention may be utilized in pharmaceutical compositions for the prevention and treatment of osteoporosis.

In vitro exposure of mononuclear leukocytes from healthy adult donors to respiratory viruses such as influenza or respiratory syncytial virus induces the production of HRF, which may induce basophil degranulation, leading to virus-induced bronchospasm (Chonmaitree et al., J Infect Dis, 164 (3), 592-4, 1991). Thus, the HRF antibody of the present invention may be utilized in pharmaceutical compositions for the prevention and treatment of respiratory diseases caused by viruses.

The pharmaceutical composition may further comprise one or more pharmaceutically acceptable carriers, diluents. For example, the pharmaceutical composition may include a pharmaceutically acceptable carrier and may be formulated for human or veterinary use and administered by various routes. The route of administration may be oral, intraperitoneal, intravenous, intramuscular, subcutaneous, intradermal, or the like. Preferably, the pharmaceutical composition is administered by formulation as an injection. The injection may be manufactured using aqueous solvents such as saline solution, Ringer's solution, non-aqueous solvents such as vegetable oils, higher fatty acid esters (e. g., ethyl oleate, etc.), alcohols (e.g., ethanol, benzyl alcohol, propylene glycol, glycerin, etc.), and may contain pharmaceutical carriers such as stabilizers to prevent deterioration (e.g., ascorbic acid, sodium bisulfite, sodium pyrosulfite, BHA, tocopherol, EDTA, etc.), emulsifiers, buffers to adjust pH, and preservatives to inhibit microbial growth (e.g., phenylmercuric nitrate, thimerosal, benzalkonium chloride, phenol, cresol, benzyl alcohol, etc.). The pharmaceutical composition may be administered in a pharmaceutically effective amount. Here, the “pharmaceutically effective amount” means an amount sufficient to exhibit a preventive or therapeutic effect and an amount that does not cause side effects or serious or excessive immune responses, and the exact administration concentration may be easily determined by those skilled in the art according to factors well known in the medical field such as the patient's age, weight, health, sex, patient's sensitivity to drugs, administration route, administration method, etc., and may be administered once or several times. In general, 0.1 mg to 100 mg per 1 kg of body weight, preferably 0.5 mg to 10 mg, may be administered at intervals of 2 or 4 weeks. However, since the dosage may increase or decrease depending on the route of administration, severity of the disease, gender, weight, age, etc., the scope of the present invention is not limited in any way by the above dosage.

Further, the present invention provides a composition for diagnosing HRF-related diseases, comprising the antibody or antigen-binding fragment thereof.

The antibody or antigen-binding fragment thereof and the HRF-related disease are as described above.

Further, the present invention provides a kit for diagnosing HRF-related diseases, comprising the antibody or antigen-binding fragment thereof.

The antibody or antigen-binding fragment thereof and the HRF-related disease are as described above. In addition, the kit for diagnosing HRF-related diseases may further comprise a composition, solution or device having one or more other components suitable for the analysis method.

Further, the present invention provides a kit for detecting HRF-related diseases, comprising the composition comprising the antibody or antigen-binding fragment thereof.

The antibody or antigen-binding fragment thereof and the HRF-related disease are as described above. In addition, the kit may further comprise a composition, solution or device having one or more other components suitable for the analysis method.

In addition, the present invention provides a method for providing information for HRF-related diseases, comprising detecting HRF protein through an antigen-antibody reaction in an isolated biological sample of an individual suspected of having HRF-related diseases, using the antibody or antigen-binding fragment thereof.

The biological sample may be selected from the group consisting of cells, tissues, body fluids (e.g., blood, serum, lymph, etc.) obtained from a patient to be diagnosed, and may be isolated from a living body. The individual may be selected from mammals, including primates including humans, monkeys, etc., rodents including mice, rats, etc.

The antigen-antibody reaction may be performed using various methods known in the art. For example, the antigen-antibody reaction may be measured through conventional enzymatic reaction, fluorescence, luminescence and/or radiation detection, and specifically, may be measured by a method selected from the group consisting of immunochromatography, immunohistochemistry, enzyme linked immunosorbent assay (ELISA), radioimmunoassay (RIA), enzyme immunoassay (EIA), fluorescence immunoassay (FIA), luminescence immunoassay (LIA), western blotting, microarray, surface plasmon resonance (SPR), etc., but is not limited thereto.

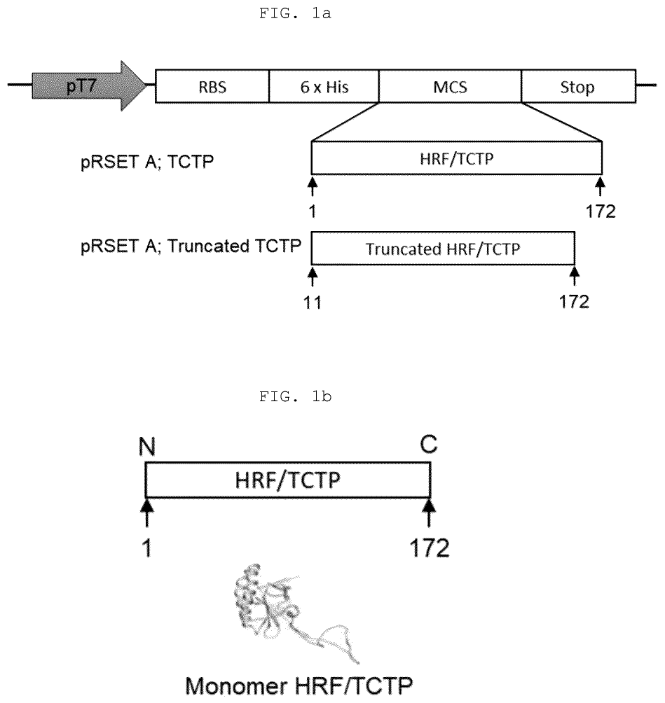

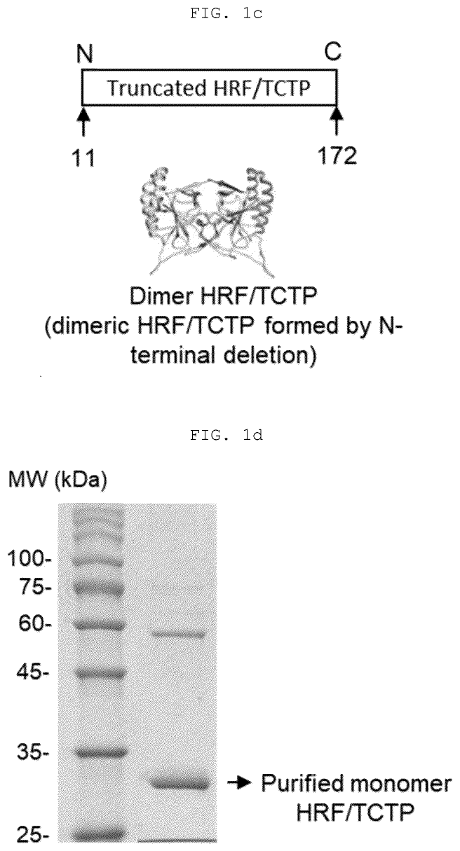

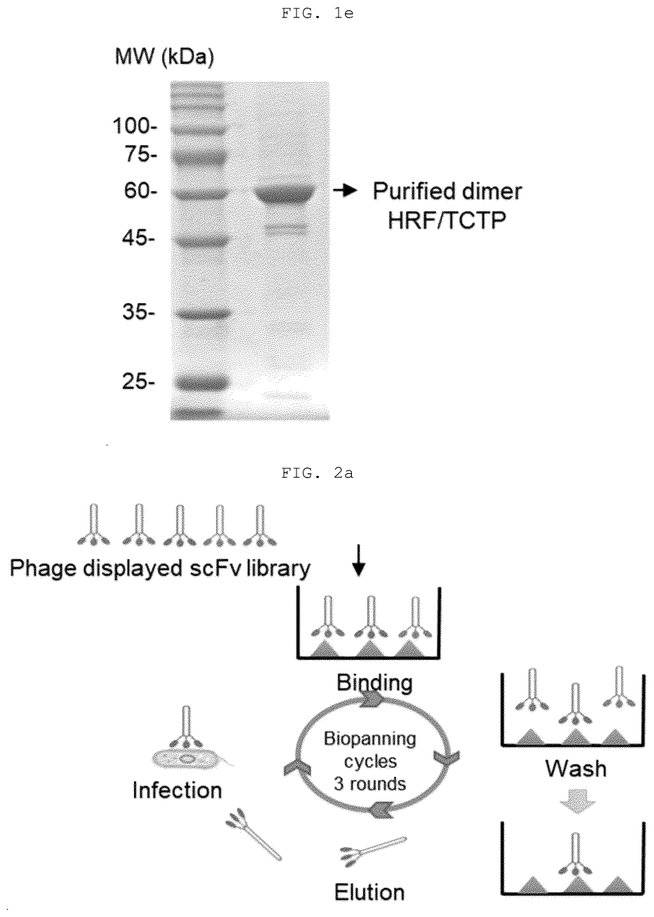

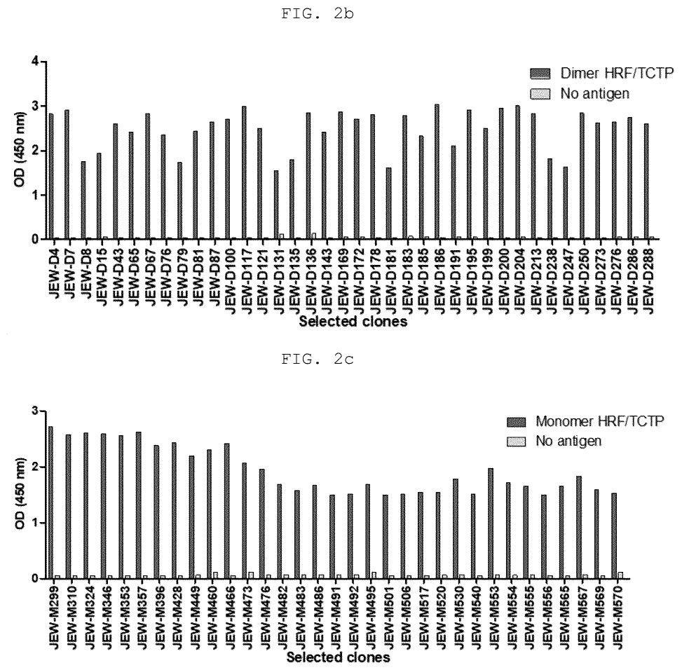

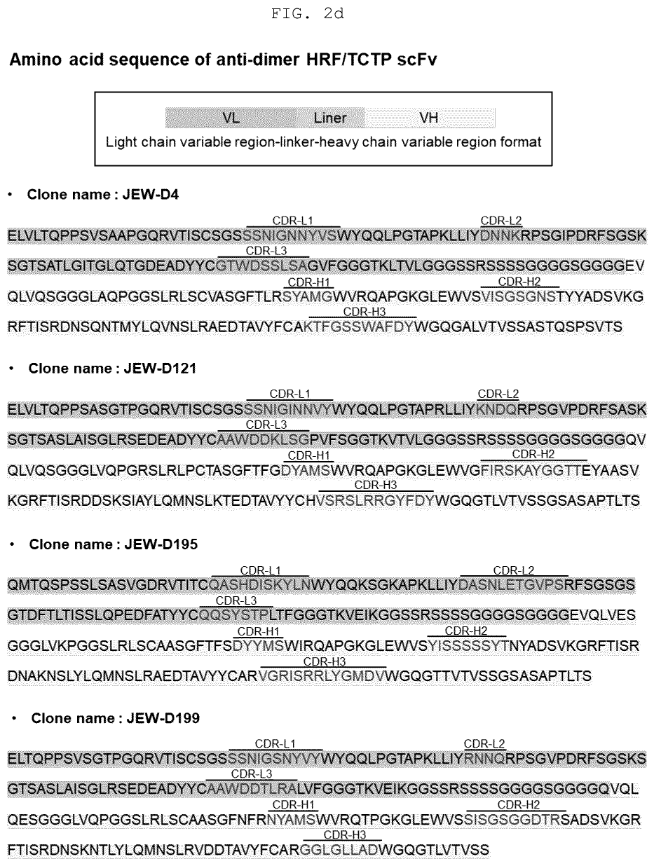

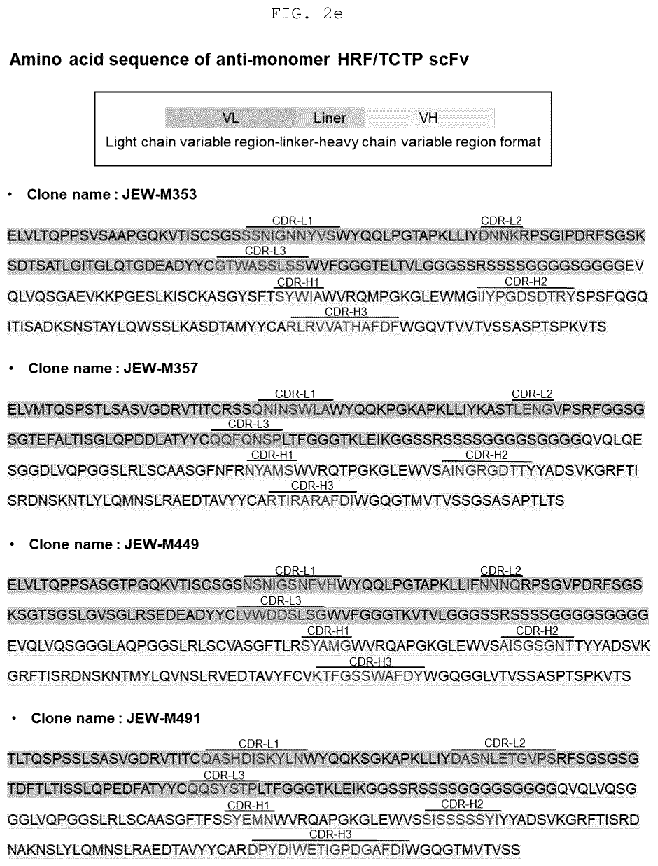

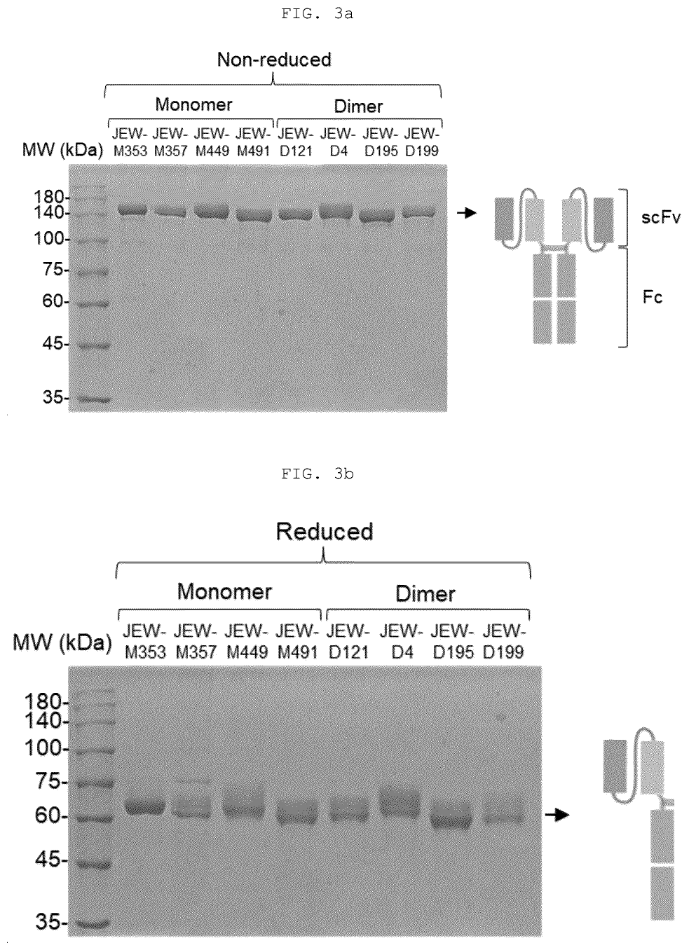

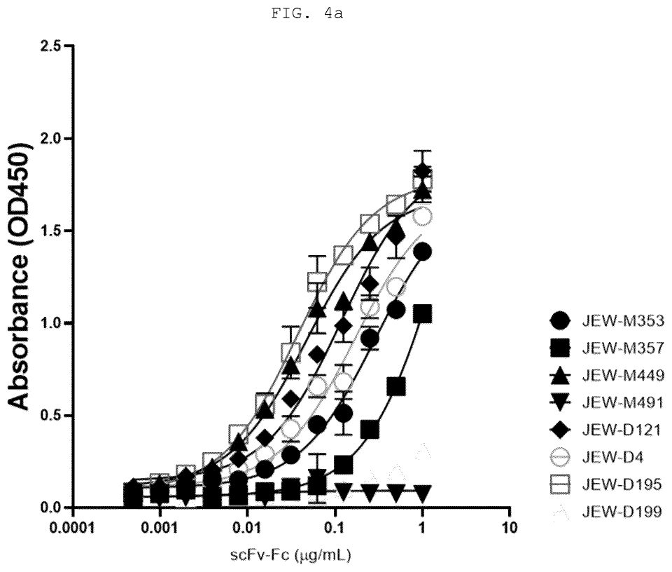

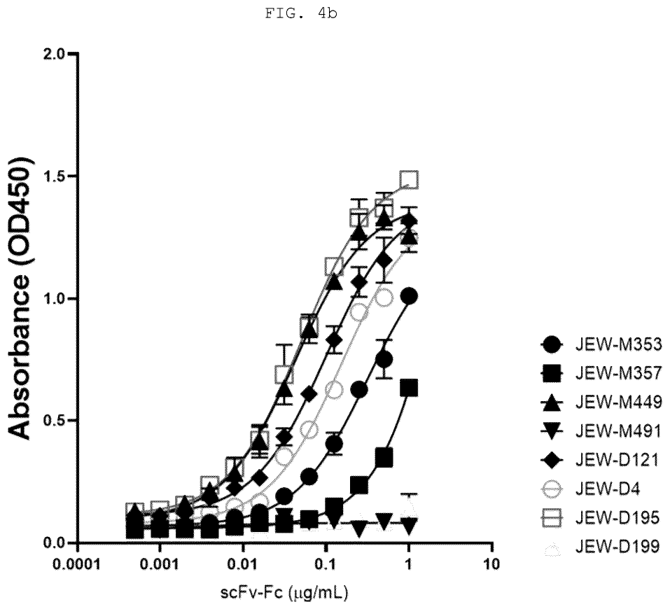

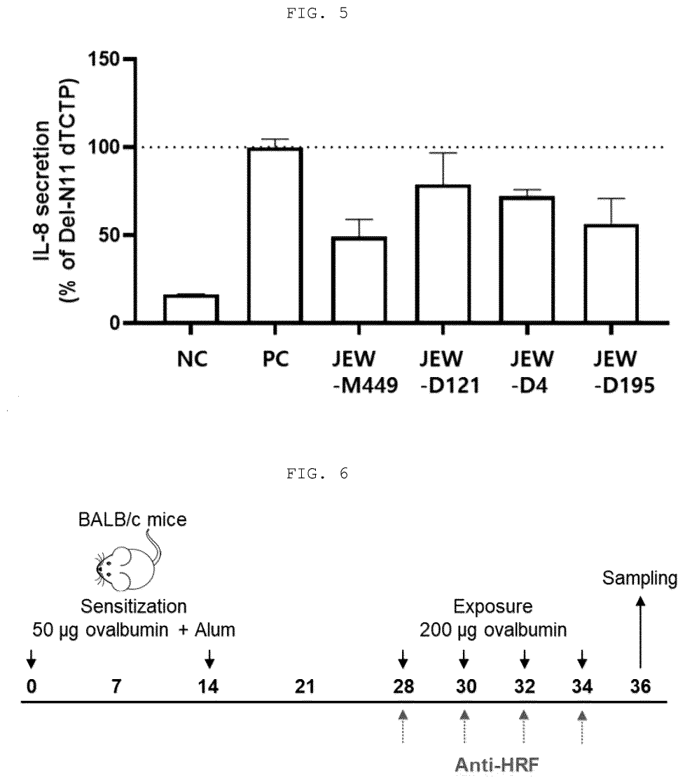

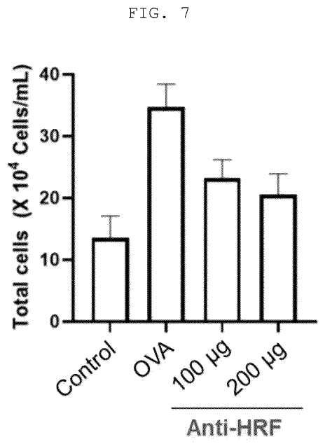

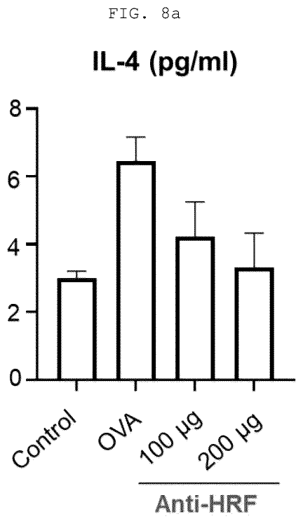

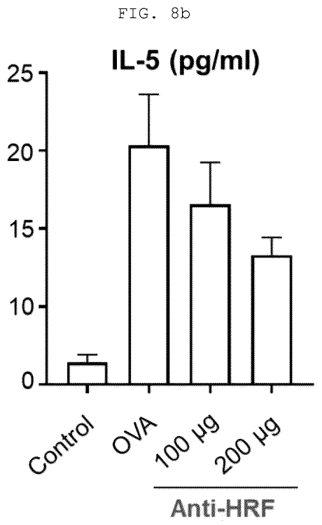

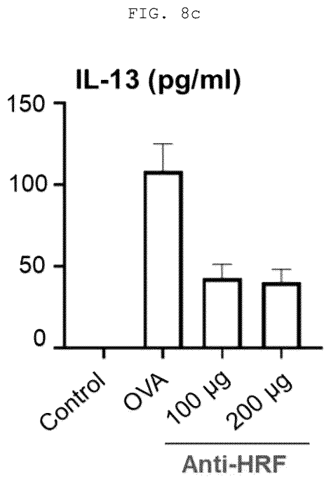

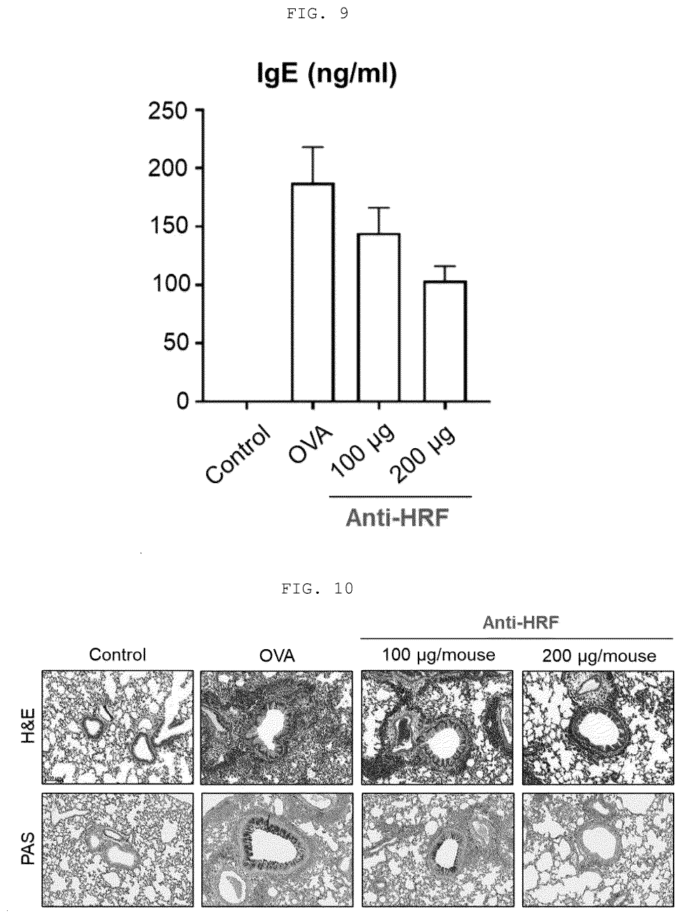

In specific Examples and Experimental Examples of the present invention, HRF proteins as monomers and/or dimers were synthesized, separated and purified (see FIGS. 1a to 1e). Using a human antibody scFv phage library, phages that specifically bind to the monomeric and/or dimeric HRF were obtained (see FIG. 2a), and 38 clones with high binding affinity to the HRF dimer and 33 clones with high binding affinity to the HRF monomer were selected (see FIG. 2b and 2c). In addition, the amino acid sequence of the scFv antibody of the clone selected above was confirmed (see FIGS. 2d and 2e), and the antibody was produced in the form of SCFV-FC (FIGS. 3a to 3b). As a result of identifying antibodies with excellent antigen-binding affinity among the above-produced anti-HRF scFv-Fc antibodies, the binding affinity to human HRF dimer was high in the order of JEW-D195≥ JEW-M449>JEW-D121>JEW-D4>JEW-M353>JEW-M357>JEW-D199>JEW-M491, and the binding affinity to mouse/rat HRF dimer was high in the order of JEW-M449≥ JEW-D195>JEW-D121>JEW-D4>JEW-M353>JEW-M357>JEW-D199>JEW-M491 (see FIGS. 4a and 4b). In addition, BEAS-2B cells, a human bronchial epithelial cell line, were treated with HRF dimer to induce BEAS-2B cell activation, and then treated with the anti-HRF monoclonal antibody of the present invention, thereby confirming that IL-8, an inflammatory mediator, was inhibited (see FIG. 5). In addition, it was confirmed in asthma and rhinitis animal models that administration of the anti-HRF SCFv-Fc antibody of the present invention showed a decrease in total leukocyte count (see FIG. 7), a decrease in IL-4, IL-5, and IL-13 levels (see FIG. 8a to FIG. 8c), a decrease in ovalbumin-specific IgE production amount in plasma (see FIG. 9), and a decrease in inflammatory cell infiltration and mucus secretion (see FIG. 10). Therefore, it can be seen that the anti-HRE antibody of the present invention has an effect of inhibiting inflammation, and may be usefully employed as a treatment agent for HRF-related diseases.

### MODE FOR CARRYING OUT THE INVENTION

Hereinafter, the present invention will be described in more detail through Examples.

These Examples are only provided for illustrating the present invention, but the scope of the present invention is not limited by these Examples.

### <Example 1> Expression, Isolation, and Purification of HRF

Experiments were Performed to Produce HRF Monomer or Dimer.

Specifically, as shown in FIG. 1a, to produce HRF dimer, an expression vector was constructed so that 10 amino acids at the N-terminus were deleted. Then, the pRSET A (Invitrogen) expression vector with the HRF gene inserted was transformed into E. coli BL21 (DE3) pLysS (Promega) and cultured to induce the synthesis of recombinant proteins (FIG. 1b and FIG. 1c). The culture medium was collected by eluting the His-tagged HRF protein bound to Ni²⁺ using His. Bind resin (Novagen) with an elution buffer. Secondary protein purification was performed to increase the purification of recombinant HRF isolated and purified by His-tag column. The secondary protein purification was performed by repurification using an anion exchange column, Mono Q HR 5/5 column (Amersham Pharmacia Biotech). In more detail, the Mono Q HR 5/5 column was washed by flowing triple distilled water for about 5 minutes, followed by the introduction of 20 mM Tris-HCl, 50 mM NaCl, and 1 mM EDTA (pH 7.4) for about 40 minutes to stabilize the column. The proteins from the previous primary purification were loaded onto the column and then 0 to 400 mM NaCl was passed through the column to collect samples at 1 ml/min for each concentration gradient. These fractions were subjected to SDS-PAGE, followed by coomassie brilliant blue staining to select only the fractions eluting HRF (FIG. 1d and FIG. 1e). The fractions were then concentrated using vivaspin (Vivascience), replacing the buffer composition with PBS.

### <Example 2> Screening of Specific Antibodies by Panning

Panning experiments were performed using a human antibody, single-chain variable fragment (scFv) phage library to enrich phages that specifically bind to the HRF antigen.

Specifically, 5 μg of HRF antigen was added to 2 ml of PBS, then placed in an immunotube, and left overnight at 4° C. to adsorb the antigen on the surface of the test tube. The next day, the phages were washed three times with phosphate buffered saline-Tween20 (PBS-T) and blocked for 2 hours at room temperature using 3% skim milk diluted in PBS. And then, the phages were washed three times with PBS-T. To screen for phages that bind to the antigen, 3 ml of a solution containing 1012 PFU of the antibody phage library was added and reacted for 2 hours, and then the solution in the test tube was discarded and the tube was washed 10 times with PBS-T. To elute and collect the scFv phage antibody bound to the antigen, 1 ml of 0.1 M glycine/HCl (pH 2) was added and left for 10 minutes at room temperature to elute the phages. Thereafter, the phages were then neutralized by adding 50 μl of 2 M Tris-base. This panning process was repeated three times to obtain and increase the phages that specifically bind to the antigen (FIG. 2a).

### <Example 3> Selection and Identification of Positive Clones

To select anti-HRF monoclonal antibodies with high binding affinity to HRF antigens, 200 μl of SB-carbenicillin medium was added to each well of a 96-well plate, and single colonies of ER2537 E. coli containing the scFv gene were inoculated into each well using a sterilized toothpick, followed by culturing (37° C., 4 hours), and VCSM13 helper phage were added to infect the E. coli. After helper phage infection, E. coli was centrifuged to remove uninfected helper phages, and the precipitated E. coli was suspended in 250 μl of medium and cultured at 37° C. for 16 hours. The culture medium was centrifuged, and the phage solution suspended on the upper layer was recovered and subjected to ELISA.

To identify phage clones that bind to the antigen, 100 ng of the antigen was coated onto a 96-well plate overnight. The plate coated with antigen in PBS-T was washed three times with PBS. The plate was then blocked for 1 hour at room temperature with 3% skim milk. The plate was then washed three times with PBS-T, and the phage solution was added and reacted at room temperature for 1 hour. The plate was washed with PBS-T, and anti-M13-HRF bound to HRF was added and reacted at room temperature for 1 hour. The plate was washed three times with PBS-T to remove unbound antibodies. Next, the TMB substrate was added and reacted for 10 minutes to develop color, and then 2 N hydrochloric acid solution was added to terminate the color development reaction. The absorbance at 450 nm was measured using an absorbance plate reader and evaluated.

As a result, 38 clones were selected from 288 clones that were positive for the antigen HRF dimer, as shown in FIG. 2b. In addition, as shown in FIG. 2c, 33 colonies were selected that showed a positive reaction to the antigen HRF monomer.

### <Example 4> Confirmation of Amino Acid Sequence of Anti-HRF Monoclonal Antibody

The scFv gene sequences of the positive phage clones selected in Example 3 were analyzed using an ABI Prism 3730XL genetic analyzer (Applied Biosystems) to confirm the amino acid sequences of the antibodies.

As a result, eight types of anti-HRF scFv antibodies having different amino acids were obtained, as shown in FIGS. 2d and 2e.

### <Example 5> Production and Purification of Anti-HRF Monoclonal Antibody

To convert the anti-HRF monoclonal antibody confirmed through Example 4 into a scFv-Fc form, the scFv gene was inserted into a SCFv-Fc expression vector, and then transformed into HEK293F cells, thereby inducing antibody expression. Afterwards, the culture medium was centrifuged to recover the supernatant to obtain antibodies secreted outside the cells. The obtained culture medium was filtered and the antibodies were purified using affinity chromatography, protein G column. To confirm the antibodies produced and purified, SDS-PAGE analysis was performed. More specifically, 4 μg of purified antibody was loaded onto a 10% acrylamide gel after adding reducing sample buffer and non-reducing sample buffer, respectively. The gel was then subjected to electrophoresis and the proteins were subsequently stained using Coomassie blue.

As a result, as shown in FIGS. 3a and 3b, it was confirmed that an anti-HRF monoclonal antibody with a purity of 95% or higher was produced and purified.

### <Experimental Example 1> Antigen-Binding Affinity of Anti-HRF Monoclonal Antibody

To select monoclonal antibodies with good antigen binding affinity among the anti-HRF ScFv-Fc antibodies produced and purified in Example 5, ELISA binding experiments were performed.

Specifically, the HRF antigen was dispensed into a 96-well plate at a final concentration of 1 μg/ml in coating buffer (50 mM sodium carbonate, pH 9.6), and the antigen was then coated overnight at 4° C. The plate was then washed three times with PBS-T to remove unbound antigen, and the plate was blocked with 3% skim milk for 1 hour at room temperature, and washed again three times with PBS-T. Then anti-HRF monoclonal antibody was diluted and added and reacted at room temperature for 1 hour. After 1 hour of antigen-antibody binding, the plate was washed 3 times with PBS-T to remove unbound antibodies, and then the detection antibody (HRF conjugated anti-human IgG) to which HRF was bound was added and reacted at room temperature for 1 hour. The plate was then washed five times with PBS-T to remove unbound detection antibodies. Next, the TMB substrate was added and reacted for 10 minutes to develop color, and then 2 N hydrochloric acid solution was added to terminate the color development reaction. The antibodies with good antigen binding affinity were identified by measuring the absorbance at 450 nm using an absorbance plate reader

As shown in FIG. 4a, the binding affinity to human HRF dimer was found to be high in the order JEW-D195≥JEW-M449>JEW-D121>JEW-D4>JEW-M353>JEW-M357>JEW-D199>JEW-M491. In addition, as shown in FIG. 4b, the binding affinity to mouse/rat HRF dimer was found to be high in the order JEW-M449≥JEW-D195>JEW-D121>JEW-D4>JEW-M353>JEW-M357>JEW-D199>JEW-M491.

### <Experimental Example 2> Confirmation of Efficacy in Inhibiting IL-8 Secretion of Bronchial Epithelial Cell

To confirm the in vitro efficacy of the monoclonal antibody with excellent antigen binding affinity, BEAS-2B cells, a human bronchial epithelial cell line, were used. BEAS-2B cells were treated with HRF dimer to induce activation, and after a certain period of time, the increased production of IL-8, an inflammatory mediator contained in the cell supernatant, was confirmed to test the efficacy of the produced anti-HRF monoclonal antibody.

More specifically, BEAS-2B cells were cultured in DMEM medium at 37° C., in the presence of 5% carbon dioxide, and transferred to a 48-well plate for growth for 24 hours when the cells reached approximately 80% confluency. Cells were washed twice with DMEM medium when they grew to 80-90% confluency, and each monoclonal antibody (30 μg/ml) was pre-mixed with HRF (2 μg/ml), and after 60 minutes, the mixture was treated to the cells and cultured for 18 hours. The amount of IL-8 production in the cell supernatant was quantified using a commercially available ELISA kit (Biolegend).

As a result, as shown in FIG. 5, treatment of BEAS-2B cells with HRF alone induced cell activation, resulting in increased production of the inflammatory mediator IL-8 contained in the cell supernatant. On the other hand, when anti-HRF monoclonal antibodies (JEW-M449, JEW-D121, JEW-D4, JEW-D195) were treated, it was confirmed that the amount of IL-8 production was inhibited.

### <Experimental Example 3> Anti-Inflammatory Efficacy of Anti-HRF Monoclonal Antibody in Asthma Model

To confirm the efficacy of anti-HRF ScFv-Fc antibody in disease models, asthma and rhinitis animal models were induced with chicken egg ovalbumin as shown in FIG. 6.

Specifically, 7-8 week old female BALB/c mice were used as experimental animals, and 50 μg of ovalbumin (Sigma) and 1 mg of Alum were mixed in 0.2 ml of PBS and injected intraperitoneally into the mice to induce allergic asthma. On day 14 after primary systemic sensitization, the mice were subjected to a second systemic sensitization using the same method to induce immune boosting. On days 28, 30, 32, and 34 after the primary systemic sensitization, 20 μl of PBS containing ovalbumin (200 μg) dissolved therein was administered intranasally to the mice for inhalation sensitization. To evaluate the efficacy of JEW-M449, an anti-HRF scFv-Fc antibody, the antibody dose was set to 100 and 200 μg/mouse. As shown in FIG. 6, the antibody was administered intraperitoneally 15 minutes before ovalbumin inhalation sensitization. A total of four doses were administered every other day, and the animals were sacrificed 48 hours after the last antibody administration.

In bronchoalveolar lavage fluid from an animal model of allergic asthma induced by repeated sensitization to ovalbumin, inflammatory cells, including eosinophils, and inflammatory cytokines increase. Therefore, the present inventors collected bronchoalveolar lavage fluid to determine the anti-inflammatory efficacy of the anti-HRF monoclonal antibody. Specifically, for each group, 48 hours after the last ovalbumin inhalation sensitization, mice were anesthetized by intraperitoneal injection of a mixture of Zoletil and Rompun. After anesthesia and thoracotomy, an incision was made in the tracheal area, and a 20-gauge intravascular tube catheter was inserted into the trachea. The lavage fluid was collected by injecting 0.8 ml of PBS and repeating the suction three times. The collected bronchoalveolar lavage fluid was centrifuged at 4° C., 2,000 rpm for 15 minutes, and the supernatant was stored at −70° C. The cell precipitate was resuspended in 200 μl of PBS and the inflammatory cell count, i.e., total leukocyte count, was measured. The cell precipitate was resuspended using a cytospin, smeared on a slide glass, and stained using Diff-Quick (Baxter Healthcare). Total leukocyte count was then measured and compared under a microscope.

As a result, as shown in FIG. 7, it was confirmed that the total leukocyte count increased in the allergy-induced group (OVA) compared to the normal control group (Control) and the total leukocyte count decreased in a dose-dependent manner in the antibody treatment group (Anti-HRF) compared to the allergy-induced group.

Next, the levels of IL-4, IL-5, and IL-13 were measured in the supernatant of the centrifuged bronchoalveolar lavage fluid were measured using commercially available ELISA kits (Biolegend) to determine the degree of Th2 immune response as an indicator of allergic inflammation.

As a result, as shown in FIGS. 8a to 8c, the levels of IL-4, IL-5, and IL-13 increased in the allergy-induced group compared to the normal control group, and decreased in a dose-dependent manner in the antibody treatment group compared to the allergy-induced group.

Blood was collected from mice to determine the amount of ovalbumin-specific IgE production in plasma for each group. Specifically, ˜0.5 ml of blood was collected from the heart of anesthetized mice 48 hours after the last ovalbumin inhalation sensitization and centrifuged at 4° C., 1,000×g for 15 minutes to obtain plasma. The amount of ovalbumin-specific IgE production in plasma was measured using a commercially available ELISA kit (Biolegend).

As a result, as shown in FIG. 9, ovalbumin-specific IgE in plasma was not detected in the normal control group, but was confirmed to increase in the allergy-induced group. In addition, it was confirmed that the amount of ovalbumin-specific IgE production was dose-dependently reduced in the antibody treatment group compared to the allergy-induced group.

Bronchoalveolar lavage fluid was collected from each group, and a portion of lung tissue was fixed in a 10% formalin solution. The fixed tissues were dehydrated to create paraffin blocks, sectioned at 5 μm thickness, and stained with hematoxylin & eosin (H&E) and periodic acid-Schiff (PAS). Stained lung tissue sections were examined by optical microscopy for histopathological analysis.

The results of H&E staining of lung tissue to observe the degree of inflammatory cell infiltration and PAS staining to measure the degree of mucus secretion are presented in FIG. 10. While no abnormal findings were observed in the normal control group, the allergy-induced group was confirmed to exhibit airway constriction, an increased thickness of the airway walls, and infiltration of a large number of inflammatory cells, and to have an increase in the number of PAS-positive cells. On the other hand, lung tissue staining showed that the antibody-treated group was observed to have a dose-dependent decrease in inflammatory cell infiltration and the amount of PAS-positive cells as compared to the allergy-induced group.

1. An antibody or antigen-binding fragment thereof that specifically binds to monomeric HRF (histamine-releasing factor), dimeric HRF and both, comprising: a CDR sequence of a variable light chain domain (VL) or a variable heavy chain domain (VH) selected from the group consisting of:
(i) a light chain CDR1 set forth in SEQ ID NO: 2; a light chain CDR2 set forth in SEQ ID NO: 3; a light chain CDR3 set forth in SEQ ID NO: 4; a heavy chain CDR1 set forth in SEQ ID NO: 6; a heavy chain CDR2 set forth in SEQ ID NO: 7; a heavy chain CDR3 set forth in SEQ ID NO: 8;
(ii) a light chain CDR1 set forth in SEQ ID NO: 12; a light chain CDR2 set forth in SEQ ID NO: 13; a light chain CDR3 set forth in SEQ ID NO: 14; a heavy chain CDR1 set forth in SEQ ID NO: 16; a heavy chain CDR2 set forth in SEQ ID NO: 17; a heavy chain CDR3 set forth in SEQ ID NO: 18;
(iii) a light chain CDR1 as set forth in SEQ ID NO: 22; a light chain CDR2 as set forth in SEQ ID NO: 23; a light chain CDR3 as set forth in SEQ ID NO: 24; a heavy chain CDR1 as set forth in SEQ ID NO: 26; a heavy chain CDR2 as set forth in SEQ ID NO: 27; a heavy chain CDR3 as set forth in SEQ ID NO: 28;
(iv) a light chain CDR1 set forth in SEQ ID NO: 32; a light chain CDR2 set forth in SEQ ID NO: 33; a light chain CDR3 set forth in SEQ ID NO: 34; a heavy chain CDR1 set forth in SEQ ID NO: 36; a heavy chain CDR2 set forth in SEQ ID NO: 37; a heavy chain CDR3 set forth in SEQ ID NO: 38;
(v) a light chain CDR1 set forth in SEQ ID NO: 42; a light chain CDR2 set forth in SEQ ID NO: 43; a light chain CDR3 set forth in SEQ ID NO: 44; a heavy chain CDR1 set forth in SEQ ID NO: 46; a heavy chain CDR2 set forth in SEQ ID NO: 47; a heavy chain CDR3 set forth in SEQ ID NO: 48;
(vi) a light chain CDR1 set forth in SEQ ID NO: 52; a light chain CDR2 set forth in SEQ ID NO: 53; a light chain CDR3 set forth in SEQ ID NO: 54; a heavy chain CDR1 set forth in SEQ ID NO: 56; a heavy chain CDR2 set forth in SEQ ID NO: 57; a heavy chain CDR3 set forth in SEQ ID NO: 58;
(vii) a light chain CDR1 set forth in SEQ ID NO: 62; a light chain CDR2 set forth in SEQ ID NO: 63; a light chain CDR3 set forth in SEQ ID NO: 64; a heavy chain CDR1 set forth in SEQ ID NO: 66; a heavy chain CDR2 set forth in SEQ ID NO: 67; a heavy chain CDR3 set forth in SEQ ID NO: 68; and
(viii) a light chain CDR1 set forth in SEQ ID NO: 72; a light chain CDR2 set forth in SEQ ID NO: 73; a light chain CDR3 set forth in SEQ ID NO: 74; a heavy chain CDR1 set forth in SEQ ID NO: 76; a heavy chain CDR2 set forth in SEQ ID NO: 77; a heavy chain CDR3 set forth in SEQ ID NO: 78.

2. An antibody or antigen-binding fragment thereof that specifically binds to monomeric HRF (histamine-releasing factor), dimeric HRF and both, comprising: a combination of heavy chain variable domain and light chain variable domain selected from the group consisting of:
(i) a light chain variable region set forth in SEQ ID NO: 1 and a heavy chain variable region set forth in SEQ ID NO: 5;
(ii) a light chain variable region set forth in SEQ ID NO: 11 and a heavy chain variable region set forth in SEQ ID NO: 15;
(iii) a light chain variable region set forth in SEQ ID NO: 21 and a heavy chain variable region set forth in SEQ ID NO: 25;
(iv) a light chain variable region set forth in SEQ ID NO: 31 and a heavy chain variable region set forth in SEQ ID NO: 35;
(v) a light chain variable region set forth in SEQ ID NO: 41 and a heavy chain variable region set forth in SEQ ID NO: 45;
(vi) a light chain variable region set forth in SEQ ID NO: 51 and a heavy chain variable region set forth in SEQ ID NO: 55;
(vii) a light chain variable region set forth in SEQ ID NO: 61 and a heavy chain variable region set forth in SEQ ID NO: 65; and
(viii) a light chain variable region set forth in SEQ ID NO: 71 and a heavy chain variable region set forth in SEQ ID NO: 75.

3. The antibody or antigen-binding fragment thereof of claim 1, wherein the antigen-binding fragment is selected from the group consisting of scFv (single-chain variable fragment), Fab, Fab′, F(ab′)₂, Fd and Fv.

4. The antibody or antigen-binding fragment thereof of claim 3, wherein the antigen-binding fragment is in the form of scFv-Fc (fragment, crystallizable).

5. A polynucleotide encoding the antibody or antigen-binding fragment thereof according to claim 1.

6. An expression vector comprising the polynucleotide of claim 5.

7. A transformant into which the expression vector of claim 6 is introduced.

8. A pharmaceutical composition for preventing or treating HRF-related diseases, comprising the antibody or antigen-binding fragment thereof according to claim 1.

9. The pharmaceutical composition of claim 8, wherein the HRF-related disease is selected from the group consisting of allergic diseases, inflammatory diseases, autoimmune diseases, cancer, hypertension, malaria, viral respiratory infection diseases, and osteoporosis.

10. The pharmaceutical composition of claim 9, wherein the inflammatory disease or autoimmune disease is selected from the group consisting of asthma, bronchitis, chronic obstructive pulmonary diseases, bronchiectasis, rhinitis, atopic dermatitis, urticaria, hay fever, conjunctivitis, allergic diseases such as anaphylaxis, bronchitis, pneumonia, arthritis, nephritis, psoriasis, dermatitis, Crohn's disease, enteritis, gingivitis, arteriosclerosis, coronary arteritis, hepatitis, Behcet's disease, bladder cancer, prostatitis, pyelonephritis, glomerulonephritis, osteomyelitis, thyroiditis, uveitis, peritonitis, meningitis, pulmonary fibrosis, and rheumatoid arthritis.

11. A composition for diagnosing HRF-related diseases, comprising the antibody or antigen-binding fragment thereof according to claim 1.

12. A kit for diagnosing HRF-related diseases, comprising the composition of claim 11.

13. A kit for detecting HRF, comprising a composition comprising the antibody or antigen-binding fragment thereof according to claim 1.

14. A method for diagnosing HRF-related diseases, comprising:
detecting HRF protein through an antigen-antibody reaction in an isolated biological sample of an individual suspected of having HRF-related diseases, using the antibody or antigen-binding fragment thereof according to claim 1.


SEQUENCE LISTING (83 sequences)

SEQ ID NO: 1 - protein, 111 aa, synthetic construct
  REGION 1..111: note = JEM-M491 DNA sequence
ELVLTQPPSVSAAPGQRVTISCSGSSSNIGNNYVSWYQQLPGTAPKLLIYDNNKRPSGIPDRFSGSKSGTSATLGITGLQTGDEADYYCGTWDSSLSAGVFGGGTKLTVLG

SEQ ID NO: 2 - protein, 10 aa, synthetic construct
  REGION 1..10: note = JEW-D4 CDR-L1
SSNIGNNYVS

SEQ ID NO: 3 - protein, 4 aa, synthetic construct
  REGION 1..4: note = JEW-D4 CDR-L2
DNNK

SEQ ID NO: 4 - protein, 9 aa, synthetic construct
  REGION 1..9: note = JEW-D4 CDR-L3
GTWDSSLSA

SEQ ID NO: 5 - protein, 129 aa, synthetic construct
  REGION 1..129: note = JEW-D4 Heavy chain a.a(VH)
EVQLVQSGGGLAQPGGSLRLSCVASGFTLRSYAMGWVRQAPGKGLEWVSVISGSGNSTYYADSVKGRFTISRDNSQNTMYLQVNSLRAEDTAVYFCAKTFGSSWAFDYWGQGALVTVSSASTQSPSVTS

SEQ ID NO: 6 - protein, 5 aa, synthetic construct
  REGION 1..5: note = JEW-D4 CDR-H1
SYAMG

SEQ ID NO: 7 - protein, 8 aa, synthetic construct
  REGION 1..8: note = JEW-D4 CDR-H2
VISGSGNS

SEQ ID NO: 8 - protein, 11 aa, synthetic construct
  REGION 1..11: note = JEW-D4 CDR-H3
KTFGSSWAFDY

SEQ ID NO: 9 - protein, 258 aa, synthetic construct
  REGION 1..258: note = JEW-D4 Amino acid sequence
ELVLTQPPSVSAAPGQRVTISCSGSSSNIGNNYVSWYQQLPGTAPKLLIYDNNKRPSGIPDRFSGSKSGTSATLGITGLQTGDEADYYCGTWDSSLSAGVFGGGTKLTVLGGGSSRSSSSGGGGSGGGGEVQLVQSGGGLAQPGGSLRLSCVASGFTLRSYAMGWVRQAPGKGLEWVSVISGSGNSTYYADSVKGRFTISRDNSQNTMYLQVNSLRAEDTAVYFCAKTFGSSWAFDYWGQGALVTVSSASTQSPSVTS

SEQ ID NO: 10 - DNA, 774 bp, synthetic construct
  misc_feature 1..774: note = JEW-D4 DNA sequence
  source 1..774: mol_type = other DNA
gagctcgtgttgacgcagccgccctcagtgtctgcggccccaggacagagggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctggggtattcggcggaggcaccaagctgaccgtcctaggcggtggttcctctagatcttcctcctctggtggcggtggctcgggcggtggtggggaggtgcagctggtgcagtctgggggaggcttggcacagccgggggggtccctgagactctcctgtgtggcctctggattcacccttaggagttatgccatgggctgggtccgccaggctccagggaaggggctggagtgggtctcagttatcagtggtagtggaaatagcacatattacgcagactccgtgaagggccggttcaccatctccagagacaattcccagaacacgatgtatcttcaagtgaacagtctcagagccgaagacacggccgtttatttctgtgcgaagacgtttggcagtagctgggcctttgactattggggacagggagccctcgtcaccgtctcctcagcctccacacagagcccatccgtcactagt

SEQ ID NO: 11 - protein, 111 aa, synthetic construct
  REGION 1..111: note = JEW-D121 Light chain amino acid (VL)
ELVLTQPPSASGTPGQRVTISCSGSSSNIGINNVYWYQQLPGTAPRLLIYKNDQRPSGVPDRFSASKSGTSASLAISGLRSEDEADYYCAAWDDKLSGPVFSGGTKVTVLG

SEQ ID NO: 12 - protein, 10 aa, synthetic construct
  REGION 1..10: note = JEW-D121 LCDR-L1
SSNIGINNVY

SEQ ID NO: 13 - protein, 4 aa, synthetic construct
  REGION 1..4: note = JEW-D121 LCDR-L2
KNDQ

SEQ ID NO: 14 - protein, 9 aa, synthetic construct
  REGION 1..9: note = JEW-D121 LCDR-L3
AAWDDKLSG

SEQ ID NO: 15 - protein, 132 aa, synthetic construct
  REGION 1..132: note = JEW-D121 Heavy chain amino acid (VH)
QVQLVQSGGGLVQPGRSLRLPCTASGFTFGDYAMSWVRQAPGKGLEWVGFIRSKAYGGTTEYAASVKGRFTISRDDSKSIAYLQMNSLKTEDTAVYYCHVSRSLRRGYFDYWGQGTLVTVSSGSASAPTLTS

SEQ ID NO: 16 - protein, 5 aa, synthetic construct
  REGION 1..5: note = JEW-D121 CDR-H1
DYAMS

SEQ ID NO: 17 - protein, 11 aa, synthetic construct
  REGION 1..11: note = JEW-D121 CDR-H2
FIRSKAYGGTT

SEQ ID NO: 18 - protein, 12 aa, synthetic construct
  REGION 1..12: note = JEW-D121 CDR-H3
VSRSLRRGYFDY

SEQ ID NO: 19 - protein, 261 aa, synthetic construct
  REGION 1..261: note = JEW-D121 Amino acid sequence
ELVLTQPPSASGTPGQRVTISCSGSSSNIGINNVYWYQQLPGTAPRLLIYKNDQRPSGVPDRFSASKSGTSASLAISGLRSEDEADYYCAAWDDKLSGPVFSGGTKVTVLGGGSSRSSSSGGGGSGGGGQVQLVQSGGGLVQPGRSLRLPCTASGFTFGDYAMSWVRQAPGKGLEWVGFIRSKAYGGTTEYAASVKGRFTISRDDSKSIAYLQMNSLKTEDTAVYYCHVSRSLRRGYFDYWGQGTLVTVSSGSASAPTLTS

SEQ ID NO: 20 - DNA, 783 bp, synthetic construct
  misc_feature 1..783: note = JEW-D121 DNA sequence
  source 1..783: mol_type = other DNA
gagctcgtgctgactcagccaccttcagcgtctgggacccccgggcagagggtcaccatctcttgttctggaagcagctccaacatcggcattaataatgtatactggtaccagcagctcccaggaacggcccccagactcctcatctataagaatgatcagcgaccctcaggggtccctgaccgattctctgcctccaagtctggcacctcagcctccctggccatcagtgggctccggtccgaggatgaggctgactattattgtgcagcatgggatgacaagttgagtggtccggtattcagcggaggcaccaaggtgaccgtcctaggcggtggttcctctagatcttcctcctctggtggcggtggctcgggcggtggtgggcaggtgcagctggtgcagtctgggggaggcttggtacagccagggcggtccctgagactcccctgtacagcttctggattcacctttggtgattatgctatgagctgggtccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacaccgccgtgtattactgtcatgtttcccgctcactacgccgagggtactttgactactggggccagggaaccctggtcaccgtctcctcagggagtgcatccgccccaaccctcactagt

SEQ ID NO: 21 - protein, 105 aa, synthetic construct
  REGION 1..105: note = JEW-D195 Light chain amino acid
QMTQSPSSLSASVGDRVTITCQASHDISKYLNWYQQKSGKAPKLLIYDASNLETGVPSRFSGSGSGTDFTLTISSLQPEDFATYYCQQSYSTPLTFGGGTKVEIK

SEQ ID NO: 22 - protein, 11 aa, synthetic construct
  REGION 1..11: note = JEW-D195 CDR-H1
QASHDISKYLN

SEQ ID NO: 23 - protein, 11 aa, synthetic construct
  REGION 1..11: note = JEW-D195 CDR-H2
DASNLETGVPS

SEQ ID NO: 24 - protein, 7 aa, synthetic construct
  REGION 1..7: note = JEW-D195 CDR-H3
QQSYSTP

SEQ ID NO: 25 - protein, 132 aa, synthetic construct
  REGION 1..132: note = JEW-D195 Heavy chain amino acid
EVQLVESGGGLVKPGGSLRLSCAASGFTFSDYYMSWIRQAPGKGLEWVSYISSSSSYTNYADSVKGRFTISRDNAKNSLYLQMNSLRAEDTAVYYCARVGRISRRLYGMDVWGQGTTVTVSSGSASAPTLTS

SEQ ID NO: 26 - protein, 5 aa, synthetic construct
  REGION 1..5: note = JEW-D195 CDR-H1
DYYMS

SEQ ID NO: 27 - protein, 9 aa, synthetic construct
  REGION 1..9: note = JEW-D195 CDR-H2
YISSSSSYT

SEQ ID NO: 28 - protein, 13 aa, synthetic construct
  REGION 1..13: note = JEW-D195 CDR-H3
VGRISRRLYGMDV

SEQ ID NO: 29 - protein, 255 aa, synthetic construct
  REGION 1..255: note = JEW-D195 amino acid sequence
QMTQSPSSLSASVGDRVTITCQASHDISKYLNWYQQKSGKAPKLLIYDASNLETGVPSRFSGSGSGTDFTLTISSLQPEDFATYYCQQSYSTPLTFGGGTKVEIKGGSSRSSSSGGGGSGGGGEVQLVESGGGLVKPGGSLRLSCAASGFTFSDYYMSWIRQAPGKGLEWVSYISSSSSYTNYADSVKGRFTISRDNAKNSLYLQMNSLRAEDTAVYYCARVGRISRRLYGMDVWGQGTTVTVSSGSASAPTLTS

SEQ ID NO: 30 - DNA, 765 bp, synthetic construct
  misc_feature 1..765: note = JEW-D195 DNA sequence
  source 1..765: mol_type = other DNA
cagatgacccagtctccatcctccctgtctgcttctgtaggagacagagtcaccatcacttgccaagcgagtcacgacattagcaaatatttaaattggtatcaacagaaatcagggaaagcccctaagctcctgatctacgatgcgtccaatttggaaacaggagtcccgtcaaggttcagtggcagtggatctgggacagatttcactctcaccatcagcagtctgcaacctgaagattttgcaacttactactgtcaacagagttacagtaccccgctcactttcggcggagggaccaaggtggagatcaaaggtggttcctctagatcttcctcctctggtggcggtggctcgggcggtggtggggaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggctgtgtattactgtgcgagagttgggagaatatcccggcgcctctacggtatggacgtctggggccaagggaccacggtcaccgtctcctcagggagtgcatccgccccaaccctcactagt

SEQ ID NO: 31 - protein, 108 aa, synthetic construct
  REGION 1..108: note = JEW-D199 Light chain amino acid
ELTQPPSVSGTPGQRVTISCSGSSSNIGSNYVYWYQQLPGTAPKLLIYRNNQRPSGVPDRFSGSKSGTSASLAISGLRSEDEADYYCAAWDDTLRALVFGGGTKVEIK

SEQ ID NO: 32 - protein, 10 aa, synthetic construct
  REGION 1..10: note = JEW-D199 CDR-L1
SSNIGSNYVY

SEQ ID NO: 33 - protein, 4 aa, synthetic construct
  REGION 1..4: note = JEW-D199 CDR-L2
RNNQ

SEQ ID NO: 34 - protein, 9 aa, synthetic construct
  REGION 1..9: note = JEW-D199 CDR-L3
AAWDDTLRA

SEQ ID NO: 35 - protein, 117 aa, synthetic construct
  REGION 1..117: note = JEW-D199 Heavy chain amino acid
QVQLQESGGGLVQPGGSLRLSCAASGFNFRNYAMSWVRQTPGKGLEWVSSISGSGGDTRSADSVKGRFTISRDNSKNTLYLQMNSLRVDDTAVYFCARGGLGLLADWGQGTLVTVSS

SEQ ID NO: 36 - protein, 5 aa, synthetic construct
  REGION 1..5: note = JEW-D199 CDR-H1
NYAMS

SEQ ID NO: 37 - protein, 10 aa, synthetic construct
  REGION 1..10: note = JEW-D199 CDR-H2
SISGSGGDTR

SEQ ID NO: 38 - protein, 8 aa, synthetic construct
  REGION 1..8: note = JEW-D199 CDR-H3
GGLGLLAD

SEQ ID NO: 39 - protein, 243 aa, synthetic construct
  REGION 1..243: note = JEW-D199 amino acid sequence
ELTQPPSVSGTPGQRVTISCSGSSSNIGSNYVYWYQQLPGTAPKLLIYRNNQRPSGVPDRFSGSKSGTSASLAISGLRSEDEADYYCAAWDDTLRALVFGGGTKVEIKGGSSRSSSSGGGGSGGGGQVQLQESGGGLVQPGGSLRLSCAASGFNFRNYAMSWVRQTPGKGLEWVSSISGSGGDTRSADSVKGRFTISRDNSKNTLYLQMNSLRVDDTAVYFCARGGLGLLADWGQGTLVTVSS

SEQ ID NO: 40 - DNA, 729 bp, synthetic construct
  misc_feature 1..729: note = JEW-D199 DNA sequence
  source 1..729: mol_type = other DNA
gagctgactcagccaccctcagtgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacactttgagggccctggttttcggcggagggaccaaggtggagatcaaaggtggttcctctagatcttcctcctctggtggcggtggctcgggcggtggtgggcaggtgcagctgcaggagtcggggggaggcttggtacagcctggggggtccctgagactctcctgtgcagcctctggattcaactttaggaattatgccatgagctgggtccgccagactccagggaaggggctggagtgggtctcgtctattagtggtagtggtggtgacacaaggtccgcagactccgtgaagggccgcttcaccatctccagagacaattccaagaacacactgtatctgcaaatgaacagcctgagagtcgacgacacggctgtctatttctgtgcgagagggggattgggactactggccgactggggccaggggaccctggtcaccgtctcctca

SEQ ID NO: 41 - protein, 111 aa, synthetic construct
  REGION 1..111: note = JEW-M353 Light chain amino acid
ELVLTQPPSVSAAPGQKVTISCSGSSSNIGNNYVSWYQQLPGTAPKLLIYDNNKRPSGIPDRFSGSKSDTSATLGITGLQTGDEADYYCGTWASSLSSWVFGGGTELTVLG

SEQ ID NO: 42 - protein, 10 aa, synthetic construct
  REGION 1..10: note = JEW-M353 CDR-L1
SSNIGNNYVS

SEQ ID NO: 43 - protein, 4 aa, synthetic construct
  REGION 1..4: note = JEW-M353 CDR-L2
DNNK

SEQ ID NO: 44 - protein, 9 aa, synthetic construct
  REGION 1..9: note = JEW-M353 CDR-L3
GTWASSLSS

SEQ ID NO: 45 - protein, 130 aa, synthetic construct
  REGION 1..130: note = JEW-M353 Heavy chain amino acid
EVQLVQSGAEVKKPGESLKISCKASGYSFTSYWIAWVRQMPGKGLEWMGIIYPGDSDTRYSPSFQGQITISADKSNSTAYLQWSSLKASDTAMYYCARLRVVATHAFDFWGQVTVVTVSSASPTSPKVTS

SEQ ID NO: 46 - protein, 5 aa, synthetic construct
  REGION 1..5: note = JEW-M353 CDR-H1
SYWIA

SEQ ID NO: 47 - protein, 11 aa, synthetic construct
  REGION 1..11: note = JEW-M353 CDR-H2
IIYPGDSDTRY

SEQ ID NO: 48 - protein, 12 aa, synthetic construct
  REGION 1..12: note = JEW-M353 CDR-H3
RLRVVATHAFDF

SEQ ID NO: 49 - protein, 259 aa, synthetic construct
  REGION 1..259: note = JEW-M353 amino acid seqquence
ELVLTQPPSVSAAPGQKVTISCSGSSSNIGNNYVSWYQQLPGTAPKLLIYDNNKRPSGIPDRFSGSKSDTSATLGITGLQTGDEADYYCGTWASSLSSWVFGGGTELTVLGGGSSRSSSSGGGGSGGGGEVQLVQSGAEVKKPGESLKISCKASGYSFTSYWIAWVRQMPGKGLEWMGIIYPGDSDTRYSPSFQGQITISADKSNSTAYLQWSSLKASDTAMYYCARLRVVATHAFDFWGQVTVVTVSSASPTSPKVTS

SEQ ID NO: 50 - DNA, 777 bp, synthetic construct
  misc_feature 1..777: note = JEW-M353 DNA sequence
  source 1..777: mol_type = other DNA
gagctcgtgttgacgcagccgccctcagtgtctgcggcccctggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctgacacgtcagccaccctgggcatcaccggactccagactggggacgaggccgattattactgcggaacatgggctagcagcctgagttcctgggtgttcggcggaggcaccgagctgaccgtcctcggcggtggttcctctagatcttcctcctctggtggcggtggctcgggcggtggtggggaggtgcagctggtgcagtctggagcagaggtgaaaaagcccggggagtctctgaagatctcctgtaaggcttctggatacagctttaccagctactggatcgcctgggtgcgccagatgcccgggaaaggcctggagtggatggggatcatctatcctggtgactctgatactagatacagcccgtccttccaaggccagatcaccatctcagccgacaagtccaacagtaccgcctacctgcagtggagcagcctgaaggcctcggacaccgccatgtattactgtgcgagacttcgcgtcgtggcgacacatgcttttgatttctggggccaggtgacagtggtcaccgtctcttcagcatccccgaccagccccaaggtcactagt

SEQ ID NO: 51 - protein, 107 aa, synthetic construct
  REGION 1..107: note = JEW-M357 Light chain Amino acid
ELVMTQSPSTLSASVGDRVTITCRSSQNINSWLAWYQQKPGKAPKLLIYKASTLENGVPSRFGGSGSGTEFALTISGLQPDDLATYYCQQFQNSPLTFGGGTKLEIK

SEQ ID NO: 52 - protein, 8 aa, synthetic construct
  REGION 1..8: note = JEW-M357 CDR-L1
QNINSWLA

SEQ ID NO: 53 - protein, 4 aa, synthetic construct
  REGION 1..4: note = JEW-M357 CDR-L2
LENG

SEQ ID NO: 54 - protein, 7 aa, synthetic construct
  REGION 1..7: note = JEW-M357 CDR-L3
QQFQNSP

SEQ ID NO: 55 - protein, 128 aa, synthetic construct
  REGION 1..128: note = JEW-M357 Heavy chain Amino acid
QVQLQESGGDLVQPGGSLRLSCAASGFNFRNYAMSWVRQTPGKGLEWVSAINGRGDTTYYADSVKGRFTISRDNSKNTLYLQMNSLRAEDTAVYYCARTIRARAFDIWGQGTMVTVSSGSASAPTLTS

SEQ ID NO: 56 - protein, 5 aa, synthetic construct
  REGION 1..5: note = JEW-M357 CDR-H1
NYAMS

SEQ ID NO: 57 - protein, 9 aa, synthetic construct
  REGION 1..9: note = JEW-M357 CDR-H2
AINGRGDTT

SEQ ID NO: 58 - protein, 10 aa, synthetic construct
  REGION 1..10: note = JEW-M357 CDR-H3
RTIRARAFDI

SEQ ID NO: 59 - protein, 253 aa, synthetic construct
  REGION 1..253: note = JEW-M357 Amino acid sequence
ELVMTQSPSTLSASVGDRVTITCRSSQNINSWLAWYQQKPGKAPKLLIYKASTLENGVPSRFGGSGSGTEFALTISGLQPDDLATYYCQQFQNSPLTFGGGTKLEIKGGSSRSSSSGGGGSGGGGQVQLQESGGDLVQPGGSLRLSCAASGFNFRNYAMSWVRQTPGKGLEWVSAINGRGDTTYYADSVKGRFTISRDNSKNTLYLQMNSLRAEDTAVYYCARTIRARAFDIWGQGTMVTVSSGSASAPTLTS

SEQ ID NO: 60 - DNA, 759 bp, synthetic construct
  misc_feature 1..759: note = JEW-M357 DNA sequence
  source 1..759: mol_type = other DNA
gagctcgtgatgacacagtctccttccaccctgtctgcatctgtaggagacagagtcaccatcacttgccggtccagtcagaacattaattcctggttggcctggtatcagcagaaaccagggaaagcccctaaactcctgatctataaggcatctactttagaaaatggggtcccgtcaaggttcggcggcagtggatctgggacagagttcgctctcaccatcagcggcctgcagcctgatgatcttgcaacttattactgccaacagtttcagaattcacccctcactttcggcggagggaccaagttggagatcaaaggtggttcctctagatcttcctcctctggtggcggtggctcgggcggtggtgggcaggtgcagctgcaggagtcggggggagacctggtacagcctggggggtccctgagactctcctgtgcagcctctggattcaactttaggaattatgccatgagctgggtccgccagactccagggaaggggctggagtgggtctcagctattaatggtagaggtgataccacatactacgcagactccgtgaagggccgattcaccatctccagagacaattccaagaacacgctgtatcttcaaatgaacagcctgagagccgaggacacggccgtgtattactgtgcgagaacaatacgagcacgtgcttttgatatctggggccaagggacaatggtcaccgtctcttcagggagtgcatccgccccaaccctcactagt

SEQ ID NO: 61 - protein, 111 aa, synthetic construct
  REGION 1..111: note = JEW-M449 Light chain sequence
ELVLTQPPSASGTPGQKVTISCSGSNSNIGSNFVHWYQQLPGTAPKLLIFNNNQRPSGVPDRFSGSKSGTSGSLGVSGLRSEDEADYYCLVWDDSLSGWVFGGGTKVTVLG

SEQ ID NO: 62 - protein, 10 aa, synthetic construct
  REGION 1..10: note = JEW-M449 CDR-L1
NSNIGSNFVH

SEQ ID NO: 63 - protein, 4 aa, synthetic construct
  REGION 1..4: note = JEW-M449 CDR-L2
NNNQ

SEQ ID NO: 64 - protein, 9 aa, synthetic construct
  REGION 1..9: note = JEW-M449 CDR-L3
LVWDDSLSG

SEQ ID NO: 65 - protein, 129 aa, synthetic construct
  REGION 1..129: note = JEW-M449 Heavy chain sequence
EVQLVQSGGGLAQPGGSLRLSCVASGFTLRSYAMGWVRQAPGKGLEWVSAISGSGNTTYYADSVKGRFTISRDNSKNTMYLQVNSLRVEDTAVYFCVKTFGSSWAFDYWGQGGLVTVSSASPTSPKVTS

SEQ ID NO: 66 - protein, 5 aa, synthetic construct
  REGION 1..5: note = JEW-M449 CDR-H1
SYAMG

SEQ ID NO: 67 - protein, 8 aa, synthetic construct
  REGION 1..8: note = JEW-M449 CDR-H2
AISGSGNT

SEQ ID NO: 68 - protein, 11 aa, synthetic construct
  REGION 1..11: note = JEW-M449 CDR-H3
KTFGSSWAFDY

SEQ ID NO: 69 - protein, 258 aa, synthetic construct
  REGION 1..258: note = JEW-M449 Amino acid sequence
ELVLTQPPSASGTPGQKVTISCSGSNSNIGSNFVHWYQQLPGTAPKLLIFNNNQRPSGVPDRFSGSKSGTSGSLGVSGLRSEDEADYYCLVWDDSLSGWVFGGGTKVTVLGGGSSRSSSSGGGGSGGGGEVQLVQSGGGLAQPGGSLRLSCVASGFTLRSYAMGWVRQAPGKGLEWVSAISGSGNTTYYADSVKGRFTISRDNSKNTMYLQVNSLRVEDTAVYFCVKTFGSSWAFDYWGQGGLVTVSSASPTSPKVTS

SEQ ID NO: 70 - DNA, 774 bp, synthetic construct
  misc_feature 1..774: note = JEW-M449 DNA sequence
  source 1..774: mol_type = other DNA
gagctcgtgctgactcagccaccttcagcgtctgggacccccgggcagaaggtcaccatctcttgttctgggagcaactccaacatcggaagtaattttgtgcactggtaccagcaactcccaggaacggcccccaaactcctcatctttaataataatcagcggccctctggggtccctgaccgattctctggctccaagtctggcacctcaggctccctgggcgtcagtgggctccggtccgaggatgaggcagactattactgtctagtatgggatgacagcctgagtggttgggtgttcggcggagggaccaaggtgaccgtcctaggcggtggttcctctagatcttcctcctctggtggcggtggctcgggcggtggtggggaggtgcagctggtgcagtctgggggaggcctggcacagccgggggggtccctgagactctcctgtgtggcctctggattcaccctcaggagttatgccatgggctgggtccgccaggctccagggaaggggctggagtgggtctcagcaatcagcgggagtggtaataccacatactacgcagactccgtgaagggccggttcaccatctccagagacaattccaagaatacgatgtatcttcaggtgaatagcctcagagtcgaagacacggccgtttatttctgtgtgaagacgtttggcagtagctgggcctttgactactggggccagggaggcctcgtcaccgtctcctcagcatccccgaccagccccaaggtcactagt

SEQ ID NO: 71 - protein, 105 aa, synthetic construct
  REGION 1..105: note = JEW-M491 Light chain amino acid sequence
TLTQSPSSLSASVGDRVTITCQASHDISKYLNWYQQKSGKAPKLLIYDASNLETGVPSRFSGSGSGTDFTLTISSLQPEDFATYYCQQSYSTPLTFGGGTKLEIK

SEQ ID NO: 72 - protein, 11 aa, synthetic construct
  REGION 1..11: note = JEW-M491 CDR-L1
QASHDISKYLN

SEQ ID NO: 73 - protein, 11 aa, synthetic construct
  REGION 1..11: note = JEW-M491 CDR-L2
DASNLETGVPS

SEQ ID NO: 74 - protein, 7 aa, synthetic construct
  REGION 1..7: note = JEW-M491 CDR-L3
QQSYSTP

SEQ ID NO: 75 - protein, 126 aa, synthetic construct
  REGION 1..126: note = JEW-M491 Heavy chain amino acid sequence
QVQLVQSGGGLVQPGGSLRLSCAASGFTFSSYEMNWVRQAPGKGLEWVSSISSSSSYIYYADSVKGRFTISRDNAKNSLYLQMNSLRAEDTAVYYCARDPYDIWETIGPDGAFDIWGQGTMVTVSS

SEQ ID NO: 76 - protein, 5 aa, synthetic construct
  REGION 1..5: note = JEW-M491 CDR-H1
SYEMN

SEQ ID NO: 77 - protein, 9 aa, synthetic construct
  REGION 1..9: note = JEW-M491 CDR-H2
SISSSSSYI

SEQ ID NO: 78 - protein, 18 aa, synthetic construct
  REGION 1..18: note = JEW-M491 CDR-H3
RDPYDIWETIGPDGAFDI

SEQ ID NO: 79 - protein, 249 aa, synthetic construct
  REGION 1..249: note = JEW-M491 Amino acid sequence
TLTQSPSSLSASVGDRVTITCQASHDISKYLNWYQQKSGKAPKLLIYDASNLETGVPSRFSGSGSGTDFTLTISSLQPEDFATYYCQQSYSTPLTFGGGTKLEIKGGSSRSSSSGGGGSGGGGQVQLVQSGGGLVQPGGSLRLSCAASGFTFSSYEMNWVRQAPGKGLEWVSSISSSSSYIYYADSVKGRFTISRDNAKNSLYLQMNSLRAEDTAVYYCARDPYDIWETIGPDGAFDIWGQGTMVTVSS

SEQ ID NO: 80 - DNA, 747 bp, synthetic construct
  misc_feature 1..747: note = JEW-M491 DNA sequence
  source 1..747: mol_type = other DNA
acactcacgcagtctccatcctccctgtctgcttctgtaggagacagagtcaccatcacttgccaagcgagtcacgacattagcaaatatttaaattggtatcaacagaaatcagggaaagcccctaagctcctgatctacgatgcgtccaatttggaaacaggagtcccgtcaaggttcagtggcagtggatctgggacagatttcactctcaccatcagcagtctgcaacccgaagattttgcaacttactactgtcaacagagttacagtaccccgctcactttcggcggagggaccaagctggagatcaaaggtggttcctctagatcttcctcctctggtggcggtggctcgggcggtggtgggcaggtgcagctggtgcagtctgggggaggcttggtacagcctggagggtccctgagactctcctgtgcagcctctggattcaccttcagtagttatgaaatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatccttacgatatttgggagacaattggccccgatggcgcttttgatatctggggccaagggacaatggtcaccgtctcttca

SEQ ID NO: 81 - protein, 249 aa, synthetic construct; identical to SEQ ID NO: 79
  REGION 1..249: note = 18-mer linker

SEQ ID NO: 82 - protein, 172 aa, synthetic construct
  REGION 1..172: note = monomer HRF amino acid
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC

SEQ ID NO: 83 - protein, 162 aa, synthetic construct
  REGION 1..162: note = dimer HRF amino acid
DEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC